(19) United States
(12) Patent Application Publication
(10) Pub. No.: US 2022/0265775 A1
(43) Pub. Date: Aug. 25, 2022

(54) COMPOSITIONS AND METHODS FOR TREATING PULMONARY EDEMA OR LUNG INFLAMMATION

(71) Applicant: REGENTS OF THE UNIVERSITY OF MINNESOTA, Minneapolis, MN (US)

(72) Inventors: Timothy P. Rich, Duluth, MN (US); David H. Ingbar, Minneapolis, MN (US)

(21) Appl. No.: 17/628,691

(22) PCT Filed: Jul. 29, 2020
(86) PCT No.: PCT/US2020/044062

§ 371 Date: Jan. 20, 2022

Related U.S. Application Data
(60) Provisional application No. 62/880,760, filed on Jul. 31, 2019.

(51) Int. Cl. A61K 38/22; A61K 31/196; A61K 31/17; A61K 31/513; A61K 31/138; A61K 31/573; A61K 31/7076; A61K 38/28; A61K 31/343; A61K 31/19; A61K 9/00; A61P 11/00

(52) U.S. Cl. CPC A61K 38/22; A61K 31/196; A61K 31/17; A61K 31/513; A61K 31/138; A61K 31/573; A61K 31/7076; A61K 38/28; A61K 31/343; A61K 31/19; A61K 9/0078; A61P 11/00

(57) ABSTRACT

A pharmaceutical composition for administering directly to the pulmonary tract of a subject includes an active agent effective to increase T3 concentration in the lung of the subject and a pharmaceutically acceptable buffer, adjusted to a pH of 5.5-8.5. The active agent can include a deiodinase inhibitor, a thyroid hormone mimetic, or a thyroid hormone analog. In some embodiments, the composition can include an additional active agent. The compositions can be used to treat lung inflammation. In some embodiments, the compositions also can be used to treat pulmonary edema. The compositions can be administered to a subject by direct instillation to the pulmonary tract or inhalation directly to the pulmonary tract.

### CROSS-REFERENCE TO RELATED APPLICATION

This application claims the benefit of U.S. Provisional Patent Application No. 62/880,760, filed Jul. 31, 2019, which is incorporated herein by reference in its entirety.

### GOVERNMENT FUNDING

This invention was made with government support under HL050152 and AI057164 awarded by the National Institutes of Health. The government has certain rights in the invention.

### SUMMARY

This disclosure describes, in one aspect, a pharmaceutical composition for administering directly to the pulmonary tract (e.g., nasosinus, intratracheal, intrabronchial, or alveolar airspace) of a subject. Generally, the composition includes an active agent effective to increase T3 concentration in the lung of the subject and a pharmaceutically acceptable buffer, adjusted to a pH of 5.5-8.5. The active agent can include a deiodinase inhibitor, a thyroid hormone mimetic, or a thyroid hormone analog.

In various embodiments, the active agent can include iopanoic acid, iopanoate, ipodate, propylthiourea, propylthiouracil, 6-propylthiouracil, propranolol, D-propranolol, dexamethasone, cortisol, a glucocorticoid, amiodarone, desethlaminodarone, dronedarone, (3′),4′,4,6-(tetra)trihydroxyaurone, insulin, 3′,5′-cyclic adenosine monophosphate, butyrate, a phenolphthalein dye, or an environmental halogenated chemical.

In various embodiments, the active agent can include a thyroid hormone derivative, a thyronine, a thyronamine, a thyroacetic acid, a chemically-modified thyroid hormone, a thyroid-hormone-linked macromolecule, or a thyroid hormone receptor agonist. In some of these embodiments, the active agent can include thyroxine; 3,3′,5-triiodothyronine; 3,5-dimethyl-3′-isoprophylthyronine; 3,5-dibromo-3′-pyridazinone-L-thyronine; 3,3′,5,5′ tetraiodoacetic acid; 3-iodo-thyroacetic acid; 3,5-diiodo-L-thyronine; dextro-T4; thyronamine; 3-iodothyronamine; 3,3′,5-triiodothyronamine; 3,5-diiodothyronine; 3,5-dibromo-3′-isopropyl-L-thyronine; 3′-acetyl-3,5,diiodo-L-thyronine; a T3 conjugate, a T4 conjugate; 3,5-dimethyl-4-(4′-hydroxy-3′isopropylbenyl)-phenoxyacetic acid; a 5′-substituted analog of 3,5-dimethyl-4-(4′-hydroxy-3′isopropylbenyl)-phenoxyacetic acid; 3,5-dichloro-4-[(4-hydroxy-3-isopropylphenoxy)phenyl]acetic acid; 3,5-dimethyl-4-(4′-hydroxy-3′-benzyl) benzylphenoxyacetic acid; MGL-3196; a [1-(4-hyrodxy-benzyl)-1H-indol-5-yloxy]-acetic acid; a carboxylic acid analog; a 1-benzyl-4-aminoindole-based thyroid hormone analog; a T3-cholic acid conjugate; CGS 23425; 3,5-dibromo-3-pyridazinone-1-thyronine; (1R,4S)-4-(3-chlorophenyl)-2-((3,5-dimethyl-4-(4′-hydroxy-3′-isopropropylbenzyl)phenoxy)methyl)-2-oxido-(1,3,2)-disozaphophonane; MB07811; or a 1-benzylindole-based agonist.

In some embodiments, the composition can include a second active agent.

In some embodiments, the second active agent can include iopanoic acid, iopanoate, ipodate, propylthiourea, propylthiouracil, 6-propylthiouracil, propranolol, D-propranolol, dexamethasone, cortisol, a glucocorticoid, amiodarone, desethlaminodarone, dronedarone, (3′),4′,4,6-(tetra)trihydroxyaurone, insulin, 3′,5′-cyclic adenosine monophosphate, butyrate, a phenolphthalein dye, or an environmental halogenated chemical.

In some embodiments, the second active agent can include a thyroid hormone derivative, a thyronine, a thyronamine, a thyroacetic acid, a chemically-modified thyroid hormone, a thyroid-hormone-linked macromolecule, or a thyroid hormone receptor agonist. In some of these embodiments, the second active agent can include thyroxine; 3,3′,5-triiodothyronine; 3,5-dimethyl-3′-isoprophylthyronine; 3,5-dibromo-3′-pyridazinone-L-thyronine; 3,3′,5,5′ tetraiodoacetic acid; 3-iodo-thyroacetic acid; 3,5-diiodo-L-thyronine; dextro-T4; thyronamine; 3-iodothyronamine; 3,3′,5-triiodothyronamine; 3,5-diiodothyronine; 3,5-dibromo-3′-isopropyl-L-thyronine; 3′-acetyl-3,5,diiodo-L-thyronine; a T3 conjugate, a T4 conjugate; 3,5-dimethyl-4-(4′-hydroxy-3′isopropylbenyl)-phenoxyacetic acid; a 5′-substituted analog of 3,5-dimethyl-4-(4′-hydroxy-3′isopropylbenyl)-phenoxyacetic acid; 3,5-dichloro-4-[(4-hydroxy-3-isopropylphenoxy)phenyl] acetic acid; 3,5-dimethyl-4-(4′-hydroxy-3′-benzyl) benzylphenoxyacetic acid; MGL-3196; a [1-(4-hyrodxy-benzyl)-1H-indol-5-yloxy]-acetic acid; a carboxylic acid analog; a 1-benzyl-4-aminoindole-based thyroid hormone analog; a T3-cholic acid conjugate; CGS 23425; 3,5-dibromo-3-pyridazinone-1-thyronine; (1R,4S)-4-(3-chlorophenyl)-2-((3,5-dimethyl-4-(4′-hydroxy-3′-isopropropylbenzyl)phenoxy)methyl)-2-oxido-(1,3,2)-disozaphophonane; MB07811; or a 1-benzylindole-based agonist.

In some embodiments, the second active agent can include a glucocorticoid, a mineralocorticoid, a β-adrenergic agonist, a catecholamine, a growth factor, an inhibitor of a pro-inflammatory cytokine, an inhibitor of a pro-inflammatory chemokine, a compound that augments an anti-inflammatory cytokine, a compound that augments an anti-inflammatory chemokine, a compound that induces genetic expression of a β-adrenergic receptor, a surfactant lipid, or an apoprotein replacement therapy.

In some embodiments, the second active agent can include a salt of triiodothyronine (T3) or a salt of thyroxine (T4).

In some embodiments, the composition can be aerosolized.

In some embodiments, the composition can be nebulized.

In another aspect, this disclosure describes a pharmaceutical composition for administering directly to the lung of a subject. Generally, the composition includes two or more active agents selected from a thyroid hormone, a deiodinase inhibitor, a thyroid hormone analog, a thyroid hormone mimetic, a glucocorticoid, a mineralocorticoid, a β-adrenergic agonist, a catecholamine, a growth factor, an inhibitor of a pro-inflammatory cytokine, an inhibitor of a pro-inflammatory chemokine, a compound that augments an anti-inflammatory cytokine, a compound that augments an anti-inflammatory chemokine, a compound that induces genetic expression of a β-adrenergic receptor, a surfactant lipid, or an apoprotein replacement therapy and a pharmaceutically acceptable buffer, adjusted to a pH of 5.5-8.5.

In some embodiments, the composition can be aerosolized.

In some embodiments, the composition can be nebulized.

In another aspect, this disclosure describes a method for treating a subject having, or at risk of having inflammation of lung tissues. Generally, the method includes administering to pulmonary tract of the subject any embodiment of the pharmaceutical compositions summarized above in an amount effective to ameliorate lung inflammation.

In some embodiments, the composition is administered by intratracheal instillation.

In some embodiments, the composition is administered by inhalation of an aerosolized formulation.

In some embodiments, the composition is administered by inhalation of a nebulized formulation.

In some embodiments, the total weight of the composition administered is a lung-delivered drug dose range of 10 ng to 5 mg.

In some embodiments, the composition is administered prior to the subject manifesting any symptom or clinical sign of lung inflammation.

In some embodiments, the composition is administered after the subject manifests a symptom or clinical sign of lung inflammation.

In some embodiments, the lung inflammation is a clinical sign of acute respiratory distress syndrome (ARDS).

In some embodiments, the lung inflammation is a symptom or clinical sign of premature birth, chest trauma, congestive heart failure, lung transplant, lung cancer radiotherapy, lung cancer chemotherapy, smoking, exposure to a pollutant, hypersensitivity pneumonitis, a reactive/obstructive lung disease, aspiration chemical pneumonitis/pneumonia, pneumonia, an infection of the nasosinus, intratracheal, intrabronchial or alveolar airspace, a connective tissue disease, Wegener's granulomatosis, Good pasture disease, acute eosinophilic pneumonia, chronic eosinophilic pneumonia, medication-related lung injury, cryptogenic organizing pneumonia, Churg-Strauss syndrome, congenital lung disease, COVID-19, or structural lung disease.

In another aspect, this disclosure describes a method for treating a subject having, or at risk of pulmonary edema. Generally the method includes administering to the pulmonary tract of the subject any embodiment of the pharmaceutical compositions summarized above in an amount effective to ameliorate pulmonary edema, wherein the composition is administered directly to the pulmonary tract.

In some embodiments, the composition is administered by intratracheal instillation.

In some embodiments, the composition is administered by inhalation of an aerosolized formulation.

In some embodiments, the composition is administered by inhalation of a nebulized formulation.

In some embodiments, the total weight of the composition administered is a lung-delivered drug dose range of 10 ng to 5 mg.

In some embodiments, the composition is administered prior to the subject manifesting any symptom or clinical sign of lung inflammation.

In some embodiments, the composition is administered after the subject manifests a symptom or clinical sign of lung inflammation.

In some embodiments, the lung inflammation is a clinical sign of acute respiratory distress syndrome (ARDS).

In some embodiments, the lung inflammation is a symptom or clinical sign of premature birth, chest trauma, congestive heart failure, lung transplant, lung cancer radiotherapy, lung cancer chemotherapy, smoking, exposure to a pollutant, hypersensitivity pneumonitis, a reactive/obstructive lung disease, aspiration chemical pneumonitis/pneumonia, pneumonia, an infection of the nasosinus, intratracheal, intrabronchial or alveolar airspace, a connective tissue disease, Wegener's granulomatosis, Good pasture disease, acute eosinophilic pneumonia, chronic eosinophilic pneumonia, medication-related lung injury, cryptogenic organizing pneumonia, Churg-Strauss syndrome, congenital lung disease, COVID-19, or structural lung disease.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Immunohistochemical localization of the enzyme deiodinase-3 (D3) in lung tissue from acute respiratory distress syndrome (ARDS) patients and normal human. (A) ARDS tissue samples showed characteristic diffuse alveolar damage and proteinaceous alveolar filling in the air spaces with diffusely positive D3 staining in multiple cell types. (D) ARDS tissue samples showed hyaline membrane formation with D3-positive staining of type II alveolar pneumocytes. (G) ARDS tissue samples showed proliferation and inflammatory cells in the interstitium with D3-positive staining of spindle-shaped cells and capillary endothelium. (B, E, H) Control tissue samples from normal human lung demonstrated normal histologic architecture of the alveoli and interstitium without significant D3 staining. (C, F, I) ARDS samples processed with normal mouse serum, rather than primary antibody (as a specificity control), also demonstrated no significant D3 staining.

FIG. 2. Deiodinase-3 activity and T3 quantity in lung tissue of ARDS and normal human lungs. (A) Lung D3 enzymatic activity is elevated in early ARDS lungs. (B) Lung T3 concentration is decreased in both early and late ARDS lungs. D3 activity and total T3 concentrations were measured in post-mortem human early ARDS (n=3), late ARDS (n=5) and normal control (n=4) lungs. Data are presented as mean±SEM. Groups not sharing a common superscript are significantly different by one-way ANOVA and Tukey's multiple comparison test (P<0.05).

FIG. 3. Free T3 levels after single or dual T3 dosing protocols. Mean±SD free triiodothyronine (T3) levels by dosing groups for human patients receiving only bolus of triiodothyronine as follows: 0.05 μg/kg at 0 hours+0.1 μg/kg at three hours (open triangles), 0.2 μg/kg at 0 hours (filled circles), or 0.4 μg/kg at 0 hours (filled diamonds). The shaded box represents the normal range for serum free triiodothyronine levels.

FIG. 4. Design of FDA-approved Phase I/II clinical human trial of T3 instillation for treating ARDS.

FIG. 5. Time course of change of serum T3 concentration (mean+/−SEM) vs. time in a rat model. A single dose of T3 was administered via intratracheal instillation at a dose of 2.7 μg (˜10.0 μg/kg). Samples were analyzed for total T3 using a chemiluminescence assay. Whenever possible, mean concentrations were derived from three animals/gender/time point.

FIG. 6. Serum T3 concentration (mean+/−SEM) following intravenous or intratracheal administration of liothyronine sodium (2.7 μg in 300 μl, pH 7.5) to rats. A single dose of T3 was administered intravenously (diamonds) or via intratracheal instillation (squares). Samples were analyzed for total T3 using a chemiluminescence assay.

FIG. 7. T3 increases RLE-6TN cell survival under 95% oxygen and during recovery in normoxia. (A) T3 increases RLE-6TN cell survival under hyperoxia stress. The cells were incubated in 90% O₂ and 5% CO₂ for 72 hours in the present of T3 (10⁻⁶M for A) or RT3 (10⁻⁶ M for A) in 2% stripped FBS culture medium. (B) T3 dose curve for RLE-6TN cell survival under hyperoxia. The cells were cultured in DMEM/F12 with 10% FBS in 21% O₂ and 5% CO₂ overnight. The cells were then incubated in 90% O₂ and 5% CO₂ for 72 hours in the presence of indicated concentrations of T3 in DMEM/F12 medium supplemented with 2% stripped FBS. (C) RLE-6TN cells were incubated with/without T3 in hyperoxia for 72 hours, and cells were then transferred to room air and cultured in DMEM/F12 with 10% FBS for another 72 hours. The viable cells are counted immediately after 72-hour hyperoxia exposure. Cell viability is assessed with trypan blue. The amount of viable cells under specific conditions is presented as a percentage of the cell number from hyperoxia alone. Data are mean±s.d. of four independent experiments with *=P<0.05; **=P<0.01.

FIG. 8. T3 increases the number of RLE-6TN cells after hyperoxia injury. (A) RLE-6TN cells were exposed to hyperoxia for 24 hours, then were transferred to room air for 48 hours in 10 the presence of T3 or rT3 in DMEM/F12 medium supplemented with 2% stripped FBS. (B) RLE-6TN cells were exposed to hyperoxia for 48 hours, then were transferred to room air for 48 hours in the presence of T3 or rT3 in DMEM/F12 medium supplemented with 2% stripped FBS. In both (A) and (B), the number of viable cells under specific conditions is presented as a percentage of the cell number related to hyperoxia alone in the same experiment. Data are mean s.d. of four independent experiments with *=P<0.05; **=P<0.01.

FIG. 9. Effect of T3 on hyperoxic damage of ATII requires the Nrf2 activation. RLE-6TN cells were incubated with or without 10⁻⁶ M T3 in 90% O₂ and 5% CO₂ for 24 hours in the presence of T3 (10-6M) in DMEM/F12 medium supplemented with 2% stripped FBS. The cells were then collected for Western Blot analysis. (A) Cellular total Nrf2 protein; (B) Nuclear Nrf2.

FIG. 10. HO-1 upregulation is required for T3-increased RLE-6TN cell survival in hyperoxia. (A) T3 increases total cellular HO-1 protein under hyperoxia. The cells were incubated in 90% O₂ and 5% CO₂ for 72 hours in the present of T3 (10⁻⁶ M). The cells were then collected for Western Blot analysis. (B) The cells were incubated in 21% O₂ and 5% CO₂ for 72 hours in the present of HO-1 inhibitor Tin Protoporphyrin IX (10⁻⁶M). (C) The cells were incubated in 90% O₂ and 5% CO₂ for 72 hours in the present of T3 (10⁻⁶M), with or without Tin Protoporphyrin IX (10⁻⁶M). Cell viability is assessed with trypan blue. The number of viable cells under specific conditions is presented as a percentage of the cell number related to hyperoxia alone. Data are mean±s.d. of three independent experiments with *=P<0.05; **=P<0.01.

FIG. 11. PI3K inhibitor wortmannin blocked T3-induced cell survival in hyperoxia. The cells were incubated in 90% O₂ and 5% CO₂ for 72 hours in the presence of T3 (10⁻⁶M), wortmannin (10⁻⁶M), or both in DMEM/F12 medium supplemented with 2% stripped FBS. Cell viability is assessed with trypan blue. The number of viable cells under specific conditions is presented as a percentage of the cell number related to hyperoxia alone. Data are mean±s.d. of four independent experiments with *=P<0.05; **=P<0.01.

FIG. 12. T3-induced increase activation of Akt by phosphorylation. (A) T3 augmented phosphorylation of Akt (p-Akt). (B) The PI3K inhibitor wortmannin blocked the T3-induced increases in both Nrf2 total cellular protein and nuclear protein. (C) The PI3K inhibitor wortmannin blocked the T3-induced increase in total cellular amount of HO-1 protein.

FIG. 13. Hyperoxia decreased serum total T3 concentration. Rats were exposed to ˜ 95% of oxygen for 60 hours. T3 supplementation started after 24 hours of hyperoxia exposure. Data are presented as mean±standard deviation (SD) of independent experiments (eight rats for room air control, 10 rats for hyperoxia, six rats for T3 supplementation) ***, p<0.001. RA: room air control.

FIG. 14. Effects of T3 on hyperoxia-induced indicia. (A) T3 diminishes hyperoxia-induced increases of wet/dry lung weight ratio. (B) T3 diminishes hyperoxia-induced increases of bronchoalveolar lavage (BAL) fluid protein concentration. Data are presented as mean±SD of independent experiments (four rats from two experiments for 48-hour exposure; three rats from three experiments for 60-hour exposure). *, p<0.05; **, p<0.01. RA: room air control.

FIG. 15. T3 decreases hyperoxia-induced increase of BALF nucleated cells. Data are presented as mean±SD of independent experiments (four rats from two experiments for 48-hour exposure; four rats from three experiments for 60-hour exposure). *, p<0.05; **, p<0.01. RA: room air control.

FIG. 16. T3 decreases hyperoxia-induced myeloperoxidase (MPO) activity in lung tissue. Data are presented as mean±SD. of independent experiments (four rats from two experiments for 48-hour exposure; four rats from three experiments for 60-hour exposure). *, p<0.05; **, p<0.01. RA: room air control.

FIG. 17. T3 decreases lung neutrophils (MPO-positive cells) after hyperoxia. Lung tissues were obtained at 60 hours after hyperoxia exposure with/without T3 injections. Immunostaining was performed with primary: anti-MPO antibody. Black dots represent MPO-positive cells.

FIG. 18. T3 injection reduces morphologic hyperoxic lung injury. Rat lung tissues at 60 hours of hyperoxia exposure with/without T3 injections were stained with hematoxylin, and examined by light microscopy. (A) Control: room air; (B) Hyperoxia; (C) Hyperoxia+T3.

### DETAILED DESCRIPTION OF ILLUSTRATIVE EMBODIMENTS

This disclosure describes compositions effective for treating pulmonary edema and/or lung inflammation such as, for example, processes that occur in acute respiratory distress syndrome (ARDS). The compositions are formulated to be administered directly into the lung, whether in a liquid form or as an aerosol (e.g., for nasal administration or administration via an inhaler). This disclosure further describes methods of treating lung inflammation and/or pulmonary edema by administering a formulation directly to the nasosinus, intratracheal, intrabronchial, or alveolar space.

The compositions include an inhibitor of iodothyronine deiodinase type-III (D3) and/or a thyroid hormone analog and/or a thyroid hormone mimetic. The compositions can further include T3, T4, or another treatment for lung edema and/or lung inflammation (e.g., another treatment for ARDS). D3 is a member of a group of selenoprotein compounds that regulate homeostasis of thyroid hormone levels in both local and peripheral tissue. Thyroid hormone affects lung development, lung function, and repair of injury to lung tissues. Three types of deiodinases exist: deiodinase type I, deiodinase type II, and deiodinase type-III (D3). D3 inactivates the thyroid hormones triiodothyronine (T3) and thyroxine (T4). T3 increases alveolar fluid clearance (AFC) in alveolar epithelial cells. Conversely, reducing T3 levels in the lung e.g., when T3 is inactivated by D3—can exacerbate alveolar edema. Inhibiting D3 to limit its ability to inactivate T3 can increase the local concentration (or increase the T3 ratio), potency, and/or duration of action of T3 in the ARDS lung. This enhanced T3 effect could provide direct, local therapy for reducing lung inflammation and interstitial edema, the hallmarks of ARDS. Thus, a deiodinase inhibitor may be an effective active agent, either alone or in combination with another active agent, for instillation, inhalation, or nebulization into the ARDS lung.

As noted above, the lung is a target tissue of thyroid hormone (TH). Hypothyroidism decreases alveolar fluid clearance (AFC). T3 acts locally in the alveolar space to rapidly stimulate alveolar fluid clearance. At the cellular level, thyroid hormone status affects alveolar number, the number and size of alveolar type II pneumocyte cells, and their surfactant production. Active sodium resorption is involved in clearing pulmonary (alveolar) edema in lungs at birth, in acute lung injury (ALI), in acute respiratory distress syndrome (ARDS), and in cardiogenic edema, such as congestive heart failure.

Acute respiratory distress syndrome (ARDS) is characterized by hemorrhagic inflammatory pulmonary edema with decreased alveolar fluid clearance (AFC) and high mortality. Most patients with ARDS have reduced ability to clear alveolar edema fluid. Triiodothyronine (T3) acts on alveolar type II pneumocytes to augment their Na,K-ATPase activity, thereby promoting edema fluid clearance and augmenting oxygen diffusion into the capillaries.

T3 and T4 are inactivated by the enzyme iodothyronine deiodinase type-III (D3). One effect of D3 is, therefore, to reduce T3 levels in the lung, thereby slowing alveolar fluid clearance and exacerbating alveolar edema. A slower rate of alveolar fluid clearance is associated with higher mortality and longer requirement for support with mechanical ventilation. Thus, improving alveolar fluid clearance improves outcomes for patients with conditions associated with lung edema (e.g., ARDS). This disclosure reports that T3 concentration in the lung tissue of ARDS patients is decreased, accompanied by early elevations in D3 expression and activity in human ARDS lung tissue. Given that T3 stimulates alveolar fluid clearance, D3-induced inactivation of lung T3 contributes, at least in part, to the reduced alveolar fluid clearance, promoting persistence of alveolar flooding with fluid, and/or hypoxemia in patients with lung edema.

This disclosure therefore describes pharmaceutical compositions effective for treating alveolar edema and/or lung inflammation. Generally, the compositions include an inhibitor of D3 and/or a thyroid hormone analog and/or a thyroid hormone mimetic, in an amount effective to increase local T3 concentration and/or T3 physiologic actions in lung tissue (e.g., increasing alveolar fluid clearance). The compositions can further include T3, T4, or another therapeutic effective to increase local T3 concentration and/or T3 physiologic actions in lung tissue.

Further, this disclosure describes methods that involve administering a pharmaceutical composition directly to the nasosinus, intratracheal, intrabronchial, or alveolar airspace by, for example, spray, inhalation, nebulization, or instillation.

While described herein in the context of an exemplary embodiment in which alveolar edema and/or lung inflammation are associated with acute respiratory distress syndrome (ARDS), the compositions and methods described herein can be used to treat alveolar edema and/or inflammation of lung tissue regardless of the underlying cause of the alveolar edema or inflammation (specifically including cardiogenic and pulmonary edema not associated with ARDS). Exemplary other causes of lung inflammation or alveolar edema that are treatable using the compositions and methods described herein include, for example, premature birth, chest trauma, congestive heart failure, pre- and/or post-lung transplant, pre- and/or post-lung cancer radiotherapy or chemotherapy, pneumonia, sepsis, smoking (whether tobacco or THC), exposure to pollutants (whether environmental or occupational, e.g., asbestosis, silicosis, berylliosis, Coal Worker's, pneumoconiosis, gas exposure, thermal injury, or other pneumoconiosis), hypersensitivity pneumonitis, reactive or obstructive lung diseases (e.g., asthma, chronic bronchitis, reactive airway dysfunction syndrome, or other reactive airway diseases), aspiration chemical pneumonitis or pneumonia, pneumonia or an infection of nasosinus, intratracheal, intrabronchial or alveolar airspace (e.g., bacterial, viral, fungal), connective tissue diseases (e.g., rheumatoid arthritis, systemic lupus erythematosus, scleroderma, sarcoidosis, and other related diseases), Wegener's granulomatosis, Goodpasture's disease, acute or chronic eosinophilic pneumonia, medication-related lung injury (e.g., injury from use of amiodarone, bleomycin, busulfan, mitomycin C, methotrexate, apomorphine, nitrofurantoin, or other pneumotoxic drugs), cryptogenic organizing pneumonia, Churg-Strauss syndrome, COVID-19, or congenital or structural lung disease (e.g., cystic fibrosis, bronchiectasis).

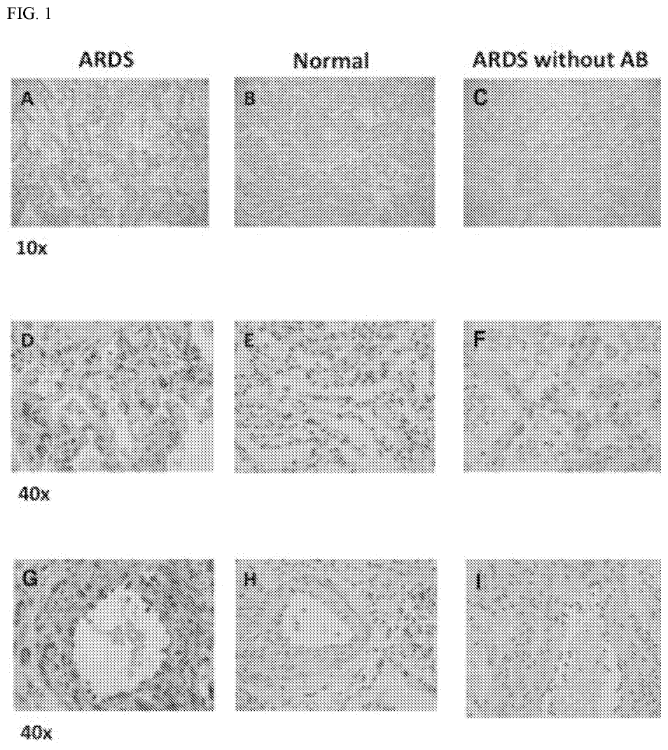

ARDS lung tissue samples showed characteristic diffuse alveolar damage with proteinaceous alveolar filling within the air spaces, hyaline membrane formation and inflammatory cells in the interstitium (FIG. 1A, 1D, 1G). Control tissue samples demonstrated normal histologic architecture of the alveoli and interstitium (FIG. 1B, 1E, 1H). Immunohistochemical localization of D3 in ARDS tissue revealed high level D3 expression in alveolar type II pneumocytes (FIG. 1A, 1D), spindle-shaped interstitial cells, and capillary endothelial cells (FIG. 1G). Normal control tissue demonstrated much less D3 antibody staining in all of these cell types (FIG. 1B, 1E, 1H). As a specificity control, removal of primary antibody resulted in no significant D3 staining of any tissue sections (FIG. 1C, 1F, 1I).

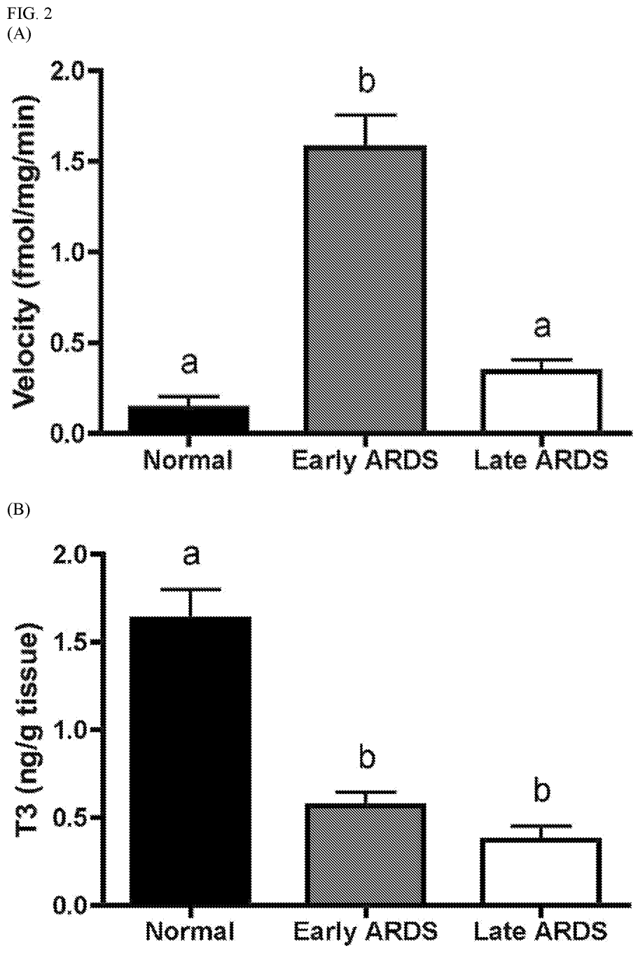

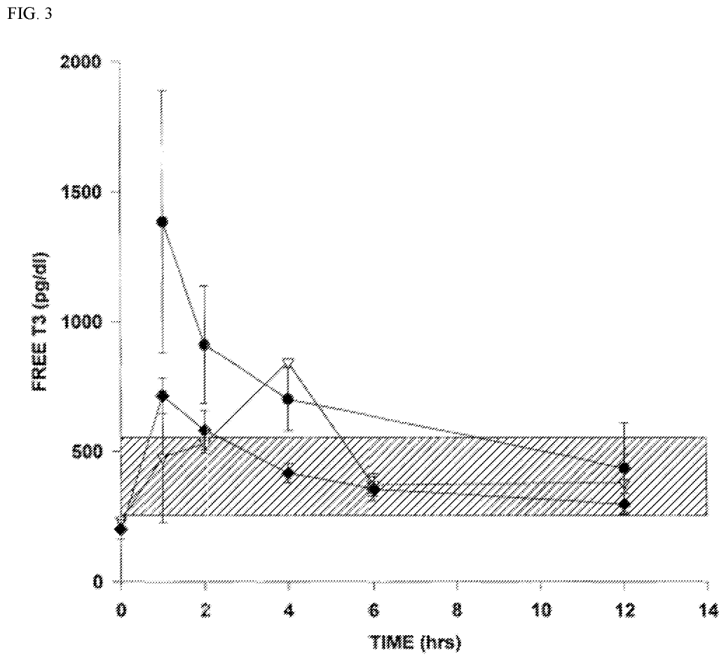

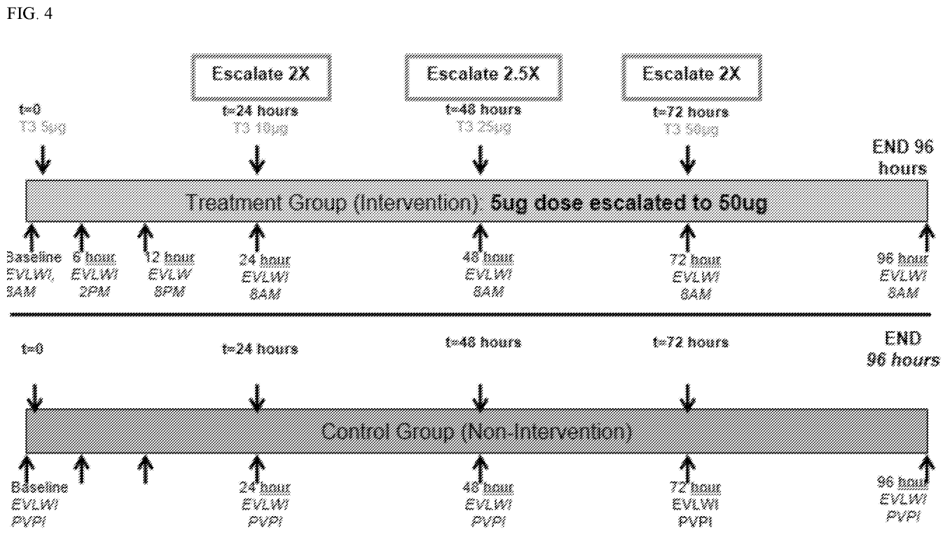

To determine whether the increase in D3 expression in ARDS lungs was associated with increased enzymatic activity, D3 enzyme activities were measured in early ARDS (n=3), late ARDS (n=5), and control (n=4) lung samples. Lung D3 enzyme activity was approximately 11.3 times higher in early ARDS versus normal control tissue (1.57 vs. 0.14+SEM fmol/mg/min, p<0.0001) (FIG. 2A). D3 activity was approximately 2.5 times higher in late ARDS versus control lungs (0.34 vs 0.14 fmol/mg/min, p=0.29, n.s.). Lung T3 levels were 65% and 77% lower in early and late ARDS, respectively, compared to control lung levels (FIG. 2B). Together, these data demonstrate that D3 expression and activity are markedly induced in the lungs of early ARDS patients and the increased D3 is associated with local reduction in total tissue T3. These data connect the role of T3 in promoting alveolar fluid clearance (AFC) in ARDS and the role of D3 causing T3 inactivation in hypoxic, inflammatory conditions.

In lung injury, the permeability of the alveolar epithelium and the capillary endothelium are increased, allowing ready transcapillary diffusion of proteins, solutes, and fluid into the interstitium and alveolar space. Resorption of interstitial edema and, particularly, alveolar edema fluid is crucial for efficient gas exchange in the alveoli. Alveolar fluid clearance is driven by active alveolar epithelial sodium resorption across the alveolar epithelial barrier through combined action of basolateral Na,K-ATPase pump and apical sodium transport proteins.

In both normal and in injured rat lungs, T3 instillation significantly increases alveolar fluid clearance. Local and/or systemic inflammation may initiate D3 induction in the ARDS lung. Acute bacterial infections and/or infarction/ischemia also can trigger D3 expression. The ARDS in the patients of this study resulted from a variety of etiologies, including pneumonia (viral or bacterial), sepsis, trauma, and post-surgical lung injury, all with inflammation as the likely common pathway to D3 induction and subsequent T3 depletion. Decreased local T3 concentration in the ARDS lung impedes alveolar fluid clearance. The decreased alveolar fluid clearance impairs oxygen diffusion and exacerbates hypoxemia, a hallmark of ARDS. At baseline in normal circumstances, five percent of total-body oxygen uptake is consumed for the mechanics of respiration and lung function. In critical illness, such as respiratory failure, the metabolic requirements of the lung usually are significantly increased to maintain adequate oxygenation and ventilation. In ARDS, systemic and local inflammation likely augment systemic and local expression of D3, lowering T3 level and downregulating lung metabolism at a time when accelerated function may be desired. Because all other organs depend on gas exchange in the lung for oxygen, and because T3 is involved in maintaining alveolar fluid clearance and diffusing capacity, T3 deficiency in the lung has a deleterious effect.

Inhibiting D3 to limit its ability to inactivate T3 could further increase the local concentration (or increase the T3 ratio), potency, physiologic effect, and/or duration of action of T3 in the lung. This enhanced T3 effect can provide direct, local therapy for reducing lung inflammation and interstitial edema. Thus, a deiodinase inhibitor may be an effective active agent, either alone or in combination with another active agent (e.g., T3, T4, a thyroid hormone analog, or a thyroid hormone mimetic), for instillation, inhalation, or nebulization into the lung.

The pharmaceutical composition can include any suitable deiodinase inhibitor. Exemplary deiodinase inhibitors include, but are not limited to, iopanoic acid (IOP), iopanoate, ipodate, propylthiourea (PTU), propylthiouracil, 6-propylthiouracil, propranolol, D-propranolol, dexamethasone, cortisol, a glucocorticoid, amiodarone, desethlaminodarone (DEA), dronedarone (Dron), (3′),4′,4,6-(tetra)trihydroxyaurone, insulin, 3′,5′-cyclic adenosine monophosphate, butyrate, phenolphthalein dyes (e.g., chlorophenol red, thymol blue, cresol red, bromocresol purple, 2-bromophenol, 2-iodophenol) or environmental halogenated chemicals (e.g., a hydroxylated PCB, a hydroxylated PBDE, an agrichemical, an antiparasitic, a pharmaceutical, or a food colorant).

Instillation of a deiodinase inhibitor, either with or without another active agent, increases the local lung T3 concentration and, therefore, augments alveolar fluid clearance in normal and hyperoxia-injured lung tissue. Hyperoxia-induced lung injury (HALI) is a well-established animal model of acute lung injury. Hyperoxia-generated reactive oxygen species (ROS) lead to alveolar epithelial and endothelial cell death by apoptosis and necrosis, contributing to lung injury. The molecular basis of oxygen toxicity is mediated by free radical ROS (reactive oxygen species) derived directly from molecular oxygen and/or derived indirectly from interactions of molecular oxygen with other species. Oxidants mediate the development of both acute and chronic lung injuries. Thyroid hormone affects antioxidant defenses of both adult and developing rat brain and lung.

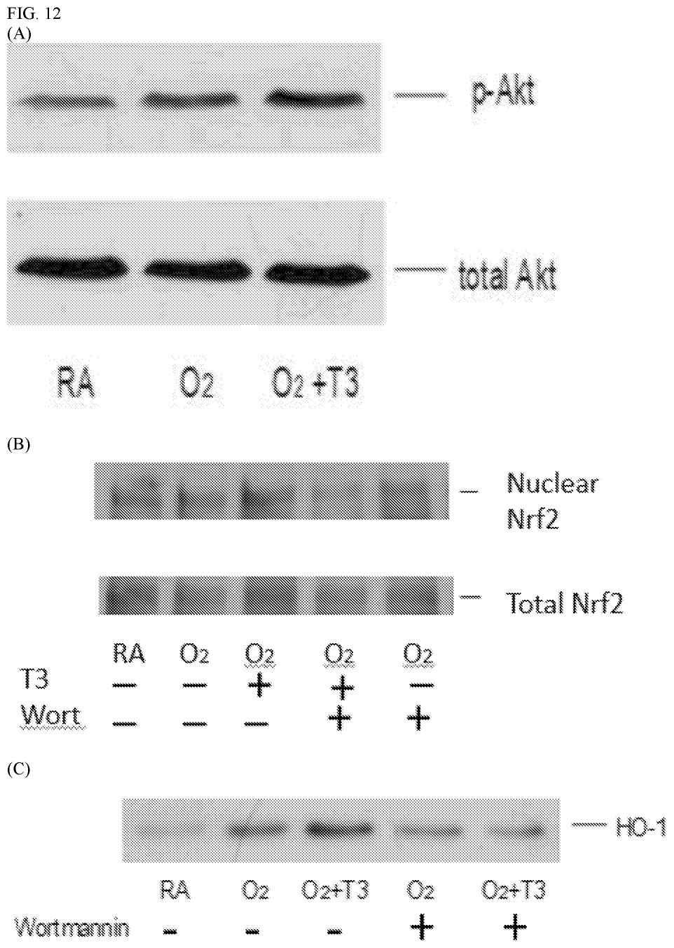

Hyperoxia decreased serum total T3 levels. Critical illness often causes the euthyroid sick syndrome or nonthyroidal illness, with decreases of serum total and free T3 concentrations. FIG. 13 shows data measuring the total serum T3 levels in rats exposed to 95% oxygen for 60 hours with or without intraperitoneal T3 supplementation (50 μg/kg bodyweight/24 hours). Hyperoxia significantly reduced the serum total T3 compared with normoxic room air (RA) rats (RA: 82.97 14.4399, hyperoxia: 53.9±11.2953, p=0.00043), and supplementation augmented serum total T3.

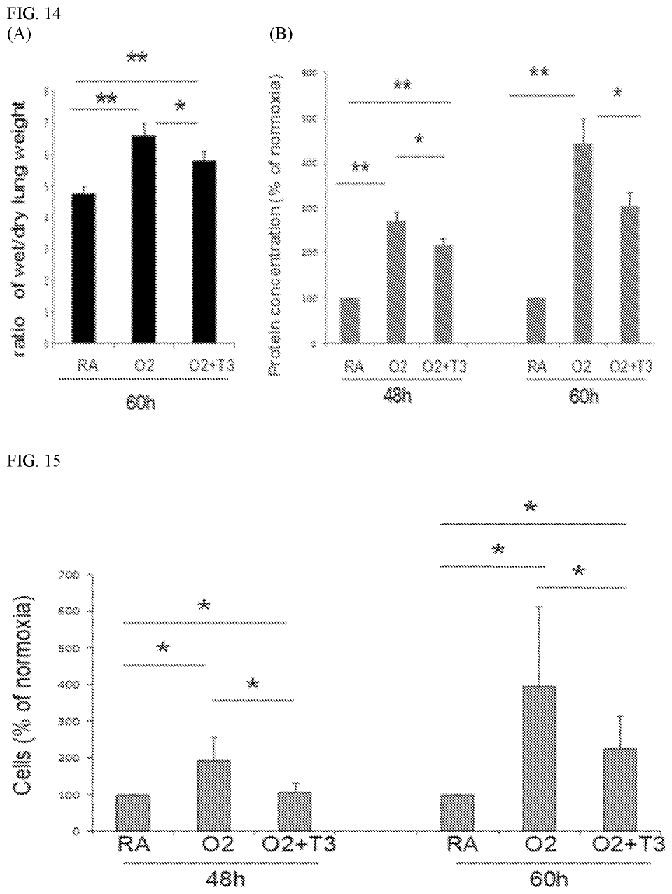

T3 decreased the hyperoxia-induced increases in lung edema and bronchoalveolar lavage fluid (BALF) protein concentration. Adult rats exposed to 95% oxygen for 60 hours have substantial lung injury as documented by increases in BALF protein concentration, permeability, and lung edema. Hyperoxia induces increased wet-to-dry lung weight ratios compared to normoxic rat lungs (6.49±0.27 vs. 5.3±0.16, respectively, p=0.004). FIG. 14A shows that 60 hours of hyperoxia again markedly increased the wet-to-dry lung weight ratio compared with room air (02 6.61±0.60 vs. RA 4.82±0.14). Treatment with intraperitoneal T3 (12.5 μg/kg body weight injected each 12 hours) significantly decreased this hyperoxia-induced increase (O₂: 6.61±0.604; O₂ with T3: 5.82±0.197, p=0.0495 vs 02 treatment) (FIG. 14A). Hyperoxia also increased markedly the BALF protein concentration at both the 48 hour and 60 hour time points, and T3 administration attenuated significantly the hyperoxic increases in the BALF protein concentration at both time points (FIG. 14B). Thus, T3 supplementation decreased the extent of pulmonary edema and the dysfunction of the alveolar epithelial barrier caused by hyperoxia.

T3 reduced the hyperoxic increases of BALF cellularity and lung tissue neutrophil accumulation. In adult rat lungs 95% oxygen exposure augmented the number of inflammatory cells in BALF. Indeed, 48 hours or 60 hours exposure to 95% oxygen markedly increased the number of bronchoalveolar lavage (BAL) cells compared to the control rats in the room air. Most of the BAL cells were mononuclear cells and macrophages, but differential cell counts were not performed. T3 administration during hyperoxia significantly reduced the BALF cell numbers at both time points compared with their hyperoxia alone counterparts (FIG. 15).

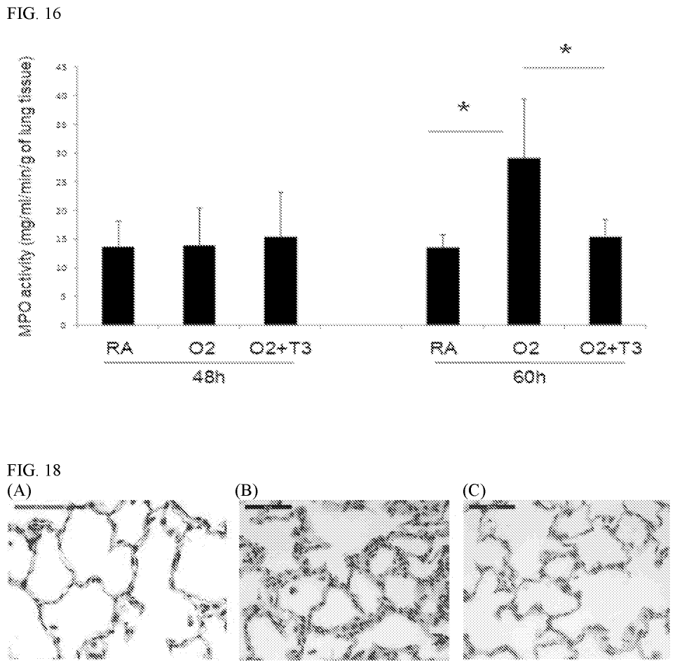

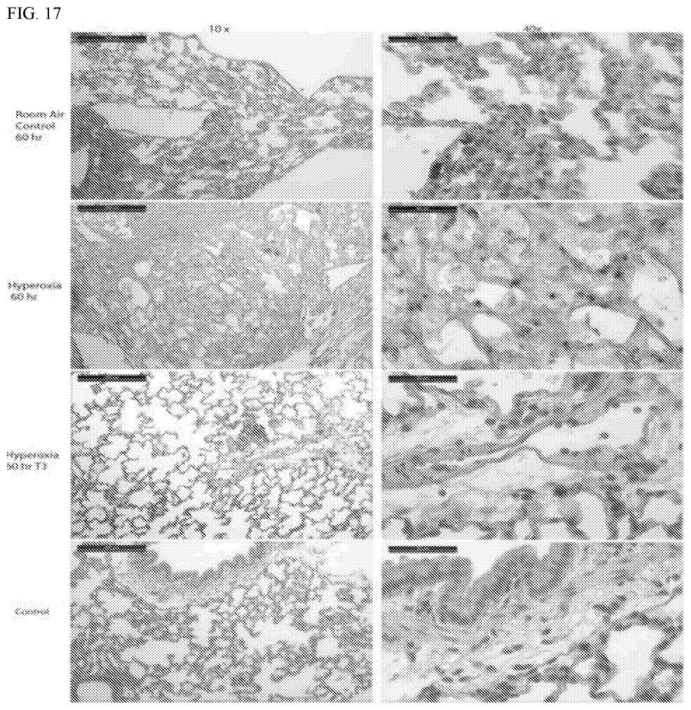

Neutrophil infiltration into the lung is a component of lung inflammation that often is a prelude to and component of lung injury. However, with T3 treatment, relatively few of the BALF cells after hyperoxia were neutrophils (data not shown). The effects of T3 on lung tissue neutrophils under hyperoxia were directly assessed in two ways: measurement of neutrophil myeloperoxidase (MPO) activity in lung homogenates and immunostaining of the lungs for MPO. In addition to being a marker of neutrophils, MPO also is an oxidizing enzyme that itself causes and amplifies lung damage. Although lung MPO activity was not altered by hyperoxia at 48 hours (FIG. 16, left panel), lung MPO activity was markedly increased at 60 hours of hyperoxia compared to room air controls (FIG. 16, right panel). T3 supplementation significantly reduced the hyperoxia-induced MPO activity (FIG. 16, right panel). Similarly, cytochemistry demonstrated that the T3-injected rats exposed to 95% oxygen for 60 hours displayed fewer MPO-positive cells in the lungs compared with hyperoxic lungs without T3 supplementation, confirming the MPO activity results (FIG. 17). Systemic T3 supplementation inhibited the hyperoxia-induced neutrophil accumulation within the lung tissue.

T3 reduced the hyperoxia-induced morphologic lung injury. Histopathological evaluation of lung sections also was performed to assess qualitatively whether T3 reduced hyperoxic lung injury. As expected, hyperoxia alone caused alveolar septal thickening, lung edema, and alveolar inflammatory cells (FIGS. 18A and 18B). In contrast, systemic T3 supplementation led to the persistence of virtually normal lung morphology (FIG. 18C). The striking difference in lung histology further demonstrated that T3 supplementation significantly attenuated hyperoxic lung injury.

Alveolar epithelial and endothelial cells maintain the integrity of the alveolar-capillary barrier and defend against oxidative injury. Prolonged exposure to hyperoxia generates excessive reactive oxygen species (ROS), damaging cells by overwhelming redox homeostasis. The nuclear factor erythroid 2-related factor 2 (Nrf2) transcription factor protects cells against oxidative insults and chemical carcinogens by coordinated transcriptional activation of a panel of antioxidant/detoxifying enzymes, including heme oxygenase-1 (HO-1), glutathione-S-transferase (GST), NAD(P)H:quinone oxidoreductase-1 (NQO-1), glutamate cysteine ligase, peroxiredoxin 3, peroxiredoxin 6, manganese superoxide dismutase, and catalase. Genetic ablation of Nrf2 enhances lung injury induced by hyperoxia, while amplification of endogenous Nrf2 activity attenuates HALL. Increased expression of antioxidant enzymes and phase 2 detoxifying enzymes in lung epithelial cells protects against the damage caused by hyperoxia-generated ROS. Nrf2-regulated HO-1 confers cytoprotection against cell death in various models of lung injury by inhibiting apoptosis. Nrf2 activation promotes alveolar cell survival during oxidative stress.

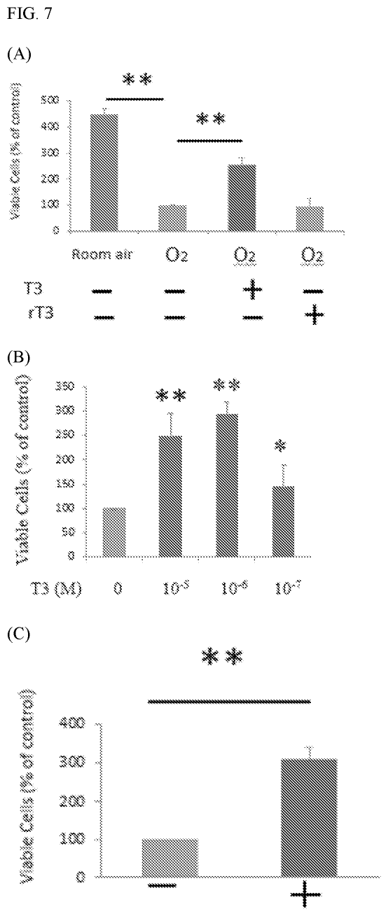

T3 increased the number of viable AT2 cells after 72 hours of exposure to 90% oxygen. In vivo hyperoxia causes rat lung inflammation and injury similar to early phase ARDS and in vitro hyperoxic exposure is a widely used model to study alveolar epithelial cell injury and function in ARDS. Using MDCK cells, cell density determined the balance of apoptosis, necrosis, and cell proliferation during hyperoxia exposure. In vivo hyperoxia exposure dramatically decreases serum T3 while T3 supplementation attenuates hyperoxia-induced lung inflammation. FIG. 7 shows that T3 protects alveolar epithelial cells from hyperoxic damage and increases their survival. Rat adult AT II-like cell line RLE-6TN were exposed to 90% oxygen for 72 hours in the presence or absence of T3 or RT3 (inactive thyroid hormone) and the number of viable cells was measured by trypan blue exclusion. Hyperoxia dramatically decreased the number of surviving AT2 cells by almost 75% compared to room air (21% oxygen) culture conditions (FIG. 7A). T3 significantly increased the number of surviving cells under hyperoxia stress by ˜2.5-fold compared with hyperoxia control. In contrast, rT3 had no role in cell survival. The protective effect of T3 was observed across a range of pharmacologic concentrations (10⁷ M to 10⁵ M) (FIG. 7B). When the AT2 cells that had been exposed to hyperoxia for 72 hours with or without exogenous T3 were allowed to recover in room air for an additional 72 hours (with no supplemental T3), the protective effect of T3 on viable AT2 cell number persisted and was of similar magnitude (FIG. 7C). The beneficial effect of having T3 at pharmacologic concentration present during hyperoxia was manifested as more surviving AT2 cells even after a recovery period in room air. These results demonstrated that T3 significantly protected AT2 cell survival during hyperoxia.

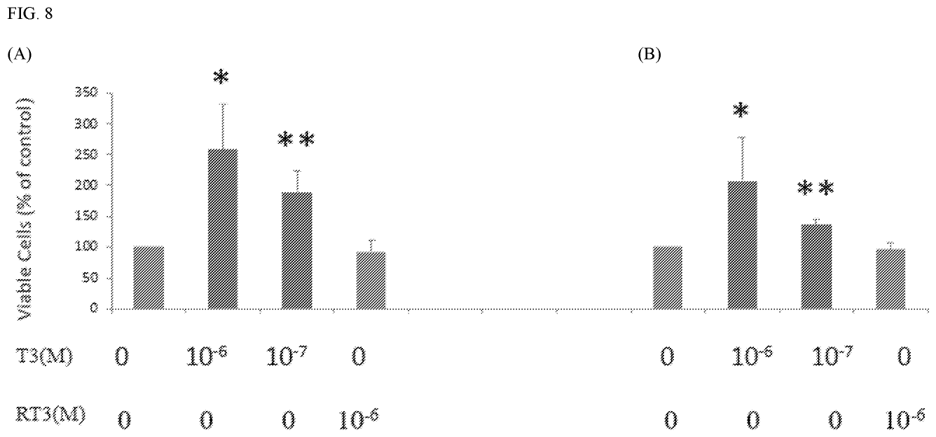

Alveolar epithelial recovery after lung inflammation and injury promotes the recovery of patients with ARDS and T3 augments this alveolar cellular recovery. FIG. 8 shows that T3 positively impacted AT2 cell recovery after hyperoxia-induced damage. RLE-6TN cells were exposed to 90% oxygen for 24 hours or 48 hours. Then the cells recovered in 21% O₂/5% CO₂ in 20 the presence or absence of T3 or rT3 for 48 hours. After either 24 or 48 hours of injury (FIG. 8A and FIG. 8B, respectively) followed by recovery, T3 significantly increased the number of viable AT2 cells at 48 hours of recovery, whereas rT3 had no protective effect. Thus T3 also has a beneficial effect on AT2 cell recovery from hyperoxia if it is present only during the recovery phase.

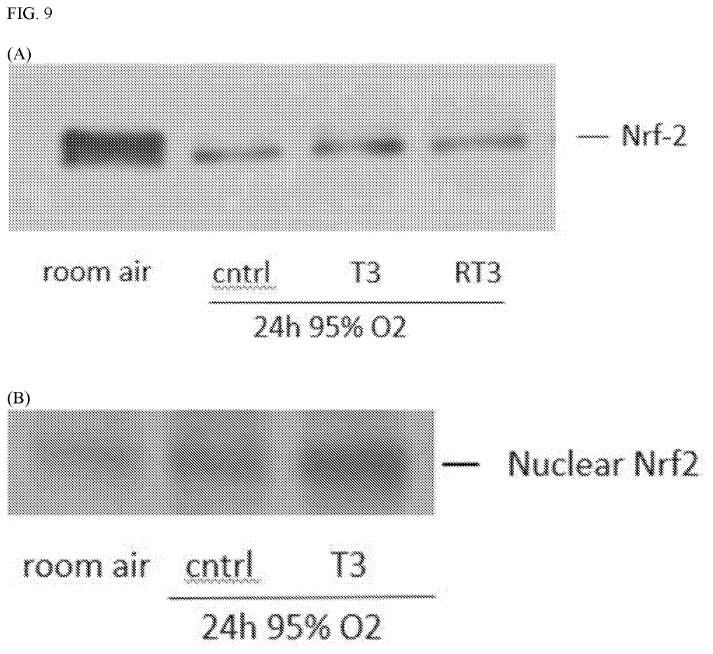

T3 increased Nrf-2 protein expression and nuclear translocation under hyperoxia stress. The transcription factor Nrf2 (NF-E2-related factor 2) promotes cellular homeostasis, especially during exposure to chemical or oxidative stress. Nrf2 regulates the basal and inducible expression of a multitude of antioxidant proteins, detoxification enzymes, and xenobiotic transporters. FIG. 9 shows that T3 increases Nrf2 activity. Total cellular and nuclear Nrf2 protein were assessed at 24 hours of hyperoxia exposure. Hyperoxia decreased the total Nrf2 protein expression, surprisingly without changing the nuclear Nrf2 protein level. T3 treatment during hyperoxia significantly increased both total cellular and nuclear Nrf-2 protein expression (FIG. 9).

T3-induced increase in HO-1 is required for T3-increased RLE-6TN cell survival in hyperoxia. Heme oxygenase-1 (HO-1) is an anti-inflammatory, antioxidative, and cytoprotective enzyme that is regulated by the activation of the major transcription factor Nrf2. HO-1 is the inducible isoform of the first and rate-limiting enzyme of heme degradation and its induction protects against oxidative stress and apoptotic cell death. Desoxyrhapontigenin upregulates Nrf2-mediated heme oxygenase-1 expression in macrophages and inflammatory lung injury. FIG. 10 shows that T3 altered HO-1 expression in AT2 cells during hyperoxia. RLE-6TN cells were exposed to 90% O₂ for 24 hours and then cell survival and HO-1 expression were measured. The impact of the HO-1 inhibitor, tin protoporphyrin, on cell survival also was determined. T3 treatment significantly enhanced total cellular protein of HO-1 during hyperoxia (FIG. 10A). Tin protoporphyrin had no effect on AT2 cell number in room air (FIG. 10B), but it blocked the T3-caused increase in cell survival and augmented cell death (FIG. 10C). Thus, T3 augments HO-1 expression during hyperoxia and HO-1 upregulation facilitates the protective effect of T3 on AT2 cell survival.

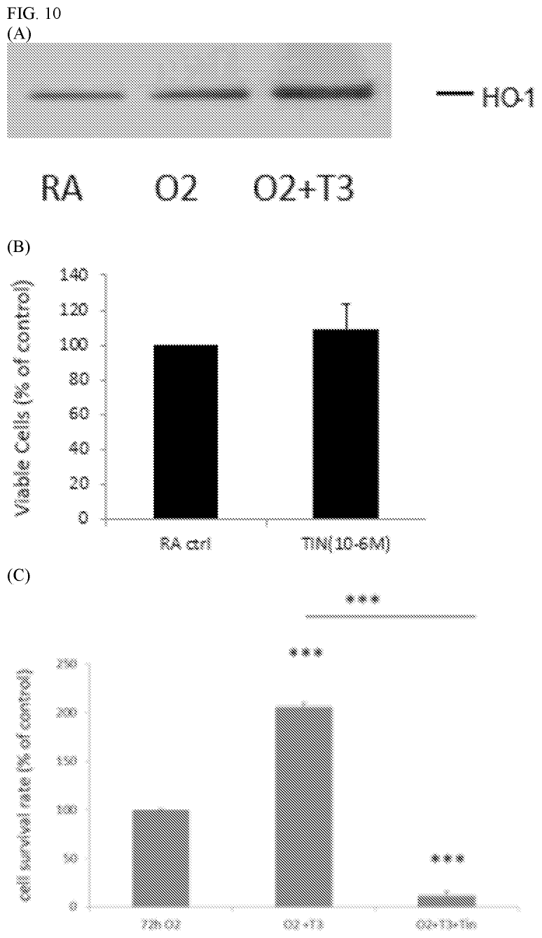

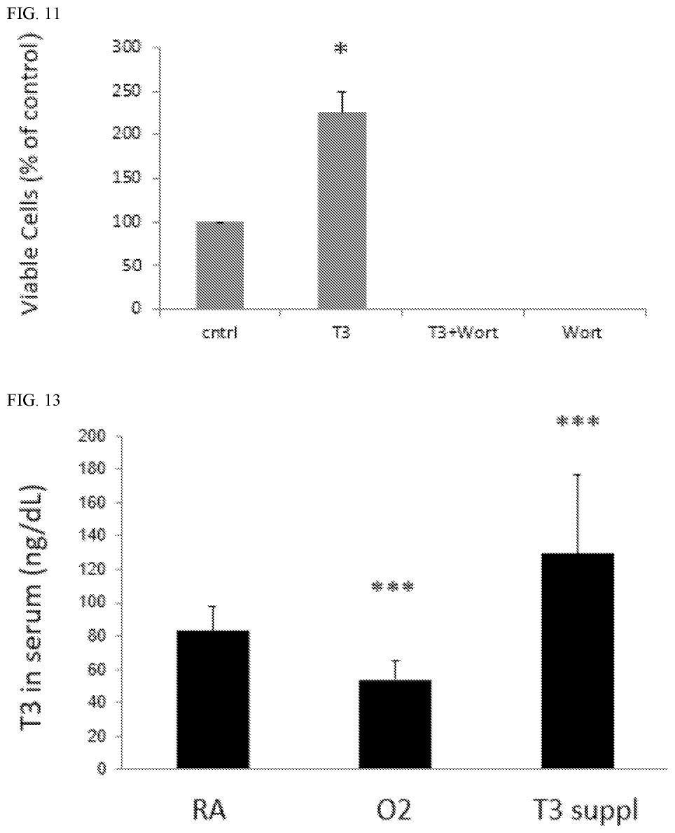

PI3-kinase activity mediates the T3 effects on AT2 cell survival, Nrf2 activity, and HO-1 expression. The PI3K/Akt is an anti-apoptotic survival pathway and is regulated by a number of receptor-dependent mechanisms. T3 stimulates PI3K activity and activation of this pathway promotes T3-induced increases of Na,K-ATPase activity and plasma membrane expression. In vascular endothelium, PI3K activation increases HO-1 expression, while PI3K activation augments Nrf2 protein levels and HO-1 activation in other cell types. To detect whether the PI3K/Akt pathway is required for the T3 protective effects on alveolar cell survival, Nrf2 activity and HO-1 protein levels during hyperoxia, RLE-6TN cells were cultured for 72 hours in hyperoxia in the presence of 10-6 M T3 and/or 100 nM wortmannin. Wortmannin blocked the T3-induced cell survival during hyperoxia, and resulted in death of almost all the cells (FIG. 11).

FIG. 12 shows the effect of PI3K on activation of Akt by phosphorylation. The RLE-6TN cells were exposed to hyperoxia for 24 hours and T3 was added for 20 minutes to some cells. Hyperoxia alone did not alter the quantity of total or phospho-Akt compared to room air control cells, whereas T3 augmented phosphorylation of Akt at Ser473 (FIG. 12A) compared with room air control and hyperoxia alone. PI3 kinase is activated by T3 during hyperoxia and activation of this pathway facilitates the protective effect of T3 on AT2 cell survival. In addition, the PI3K inhibitor wortmannin also blocked the T3-induced increases in both Nrf2 total cellular protein and nuclear protein (FIG. 12B), and total cellular amount of HO-1 protein (FIG. 12C). Thus, the PI3-kinase pathway is activated by T3 during hyperoxia and activation of this pathway is involved in the beneficial effects of T3 on the cytoprotective effectors HO-1 and Nrf2 and on AT2 cell survival.

This disclosure provides data showing that T3 at pharmacologic concentrations increases AT2 cell survival during hyperoxia and accelerated the recovery in AT2 cell number after hyperoxia. These effects were associated with activation of PI3 kinase and Nrf2 and with upregulation of HO-1 expression. The cytoprotective effects of T3 were abrogated when PI3K activation was blocked by wortmannin or when HO-1 expression was blocked by tin protoporphyrin. These findings suggest that T3 augmentation in the lung augments alveolar epithelial repair.

Thus, this disclosure provides data showing that T3 significantly decreased the severity of hyperoxia-induced lung injury, with reduced neutrophil accumulation in the lungs, diminished lung edema, and less breakdown of the alveolar epithelial permeability barrier. This disclosure also provides data showing that D3 concentrations are higher than normal in patients with ARDS when T3 levels are lower than normal. These results strongly suggest a protective anti-inflammatory effect of T3 against hyperoxic lung injury. International Publication No. WO 2019/152659 A1 describes composition of T3 for direct delivery to the pulmonary tract and methods that include administering T3 directly to the pulmonary tract. In contrast, this disclosure describes compositions and methods that aim to maintain T3 concentration in the lung by inhibiting D3 inactivation of T3.

This disclosure therefore describes compositions and methods for maintaining or restoring T3 levels in a subject. The compositions can include a D3 inhibitor, a thyroid hormone analog, a thyroid hormone mimetic, or a combination of two or more compounds from any of the foregoing classes. The compositions may further include an additional active agent such as, for example, T3 and/or T4. The composition is provided in a formulation for direct administration to the pulmonary tract, thereby limiting systemic exposure. The compositions can be effective for reducing—in some cases, eliminating-lung inflammation (e.g., associated with lung transplant, radiotherapy or chemotherapy), augmenting pulmonary edema fluid clearance, diminishing lung injury, and/or treating lung inflammation associated with pulmonary disease or injury (e.g., ARDS).

As described above, the deiodinase inhibitors can include iopanoic acid (IOP), iopanoate, ipodate, propylthiourea (PTU), propylthiouracil, 6-propylthiouracil, propranolol, D-propranolol, dexamethasone, cortisol, a glucocorticoid, amiodarone, desethlaminodarone (DEA), dronedarone (Dron), (3′),4′,4,6-(tetra)trihydroxyaurone, insulin, 3′,5′-cyclic adenosine monophosphate, butyrate, a phenolphthalein dye (e.g., chlorophenol red, thymol blue, cresol red, bromocresol purple, 2-bromophenol, 2-iodophenol) and/or an environmental halogenated chemical (e.g., a hydroxylated PCB, a hydroxylated PBDE, an agrichemical, an antiparasitic, a pharmaceutical, or a food colorant).

Exemplary thyroid hormone analogs and mimetics include, but are not limited to, a thyroid hormone derivative, a thyronine, a thyronamine, a thyroacetic acid, a chemically-modified thyroid hormone, a thyroid-hormone-linked macromolecule, or a thyroid hormone receptor agonist. Exemplary thyronines include, but are not limited to, thyroxine (T4); 3,3′,5-triiodothyronine (TRIAC); 3,5-dimethyl-3′-isoprophylthyronine (DIMIT); 3,5-dibromo-3′-pyridazinone-L-thyronine (SKF 94901); 3,3′,5,5′ tetraiodoacetic acid (TETRAC); 3-iodo-thyroacetic acid (3T1Ac); 3,5-diiodo-L-thyronine (T2); or dextro-T4 (D-T4). Exemplary thyronamines and derivatives thereof include, but are not limited to, thyronamine (TOAM); 3-iodothyronamine (T1AM); 3,3′,5-triiodothyronamine (Triam); 3,5-diiodothyronine (3,5-T2 or DIT); 3,5-dibromo-3′-isopropyl-L-thyronine (Dibit); 3′-acetyl-3,5,diiodo-L-thyronine; or a T3 or T4 sulfation or glucuronidation conjugate/metabolite. Exemplary thyroacetic acids include, but are not limited to, 3,5-dimethyl-4-(4′-hydroxy-3′isopropylbenyl)-phenoxyacetic acid (GC-1) or a related 5′-substituted analog; 3,5-dichloro-4-[(4-hydroxy-3-isopropylphenoxy)phenyl] acetic acid (KB-141); 3,5-dimethyl-4-(4′-hydroxy-3′-benzyl) benzylphenoxyacetic acid (GC-24); or MGL-3196. Chemically-modified thyroid hormones include, but are not limited to, a [1-(4-hyrodxy-benzyl)-1H-indol-5-yloxy]-acetic acid; a carboxylic acid analog (DITPA); a 1-benzyl-4-aminoindole-based TH analog; T3 conjugated to cholic acid (CGH-509A, Ciba Geigy, Basel, Switzerland); CGS 23425 (Ciba Geigy, Basel, Switzerland); or 3,5-dibromo-3-pyridazinone-1-thyronine. Exemplary thyroid hormone receptor agonists include, but are not limited to, (1R,4S)-4-(3-chlorophenyl)-2-((3,5-dimethyl-4-(4′-hydorxy-3′-isopropropylbenzyl)phenoxy)methyl)-2-oxido-(1,3,2)-disozaphophonane; MB07811 (Metabasis Therapeutics, Inc., La Jolla, Calif.); or a 1-benzylindole-based agonist such as, for example, SKL-12846 or SKL-13784 (Sanwa Kagaku Kenkyusho Co., Ltd., Nagoya, Japan).

The active agent or active agents may be formulated with any suitable pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient or is known to be injurious to lung tissue, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the active agent or active agents without causing any undesirable biological effects or interacting in a deleterious manner with any of the other components of the pharmaceutical composition in which it is contained.

The active agent or active agents may therefore be formulated into a pharmaceutical composition. The pharmaceutical compositions described herein, including those that include the D3 inhibitor, may be adjusted to neutral pH. As used herein, the term “neutral pH” refers to a pH that is pH 7.0±1.5—i.e., a pH of 5.5 to 8.5. In some embodiments, the formulation may be buffered to a minimum pH of at least 5.5, at least 6.0, at least 6.5, at least 7.0, or at least 7.5. In some embodiments, the formulation may be buffered to a maximum pH of no greater then 8.5, no greater then 8.0, no greater then 7.5, no greater than 7.0, or no greater than 6.5. In some embodiments, the formulation may be buffered to a pH that falls within a range having endpoints defined by any minimum pH listed above and any maximum pH listed above that is greater than the minimum pH. Thus, for example, the formulation may be buffered to a pH of from 5.5-8.5, such as, for example, a pH of 5.5-7.0, a pH of 6.0-8.0, a pH of 6.0-7.0, or a pH of 6.5-7.5.

The pharmaceutical composition may be formulated in a variety of forms adapted for delivery to the nasosinus, intratracheal, intrabronchial, or alveolar space. A pharmaceutical composition can be administered to a mucosal surface, such as by administration to, for example, respiratory mucosa (e.g., by spray, aerosol, nebulization, or instillation). A composition also can be administered via a sustained or delayed release. Sustained or delayed released may be accomplished through conventional, general technologies for sustained or delayed drug delivery.

The pharmaceutical composition can include a combination of two or more active agents. Exemplary active agents that may be combined with the deiodinase inhibitor to form a multicomponent pharmaceutical composition include, but are not limited to, a thyroid hormone (e.g., T3), a glucocorticoid, a mineralocorticoid, a β-adrenergic agonist (e.g., salmeterol), a catecholamine (e.g., dopamine), a growth factor (e.g., keratinocyte GF or epidermal GF), an inhibitor of a pro-inflammatory cytokine, an inhibitor of a pro-inflammatory chemokine, a compound that augments an anti-inflammatory cytokine, a compound that augments an anti-inflammatory chemokine, a compound that induces genetic expression of a β-adrenergic receptor, a surfactant lipid, or an apoprotein replacement therapy.

A pharmaceutical composition described herein may be provided in any suitable form including, but not limited to, a solution, a suspension, an emulsion, a spray, an aerosol, or any form of mixture. The pharmaceutical composition may be delivered in formulation with any pharmaceutically acceptable excipient, carrier, or vehicle. Exemplary suitable excipients include, but are not limited to, dextrose and ammonium hydroxide. For example, the formulation may be delivered in a dosage form suitable for direct delivery to the lungs such as, for example, an aerosol formulation (e.g., for nasal administration or administration via an inhaler), a non-aerosol spray, a solution, a liquid suspension, and the like. The formulation may further include one or more additives including such as, for example, an adjuvant, a colorant, a fragrance, a flavoring, and the like.

A formulation may be conveniently presented in unit dosage form and may be prepared by methods well known in the art of pharmacy. Methods of preparing a composition with a pharmaceutically acceptable carrier include the step of bringing the active agent or active agents into association with a carrier that constitutes one or more accessory ingredients. In general, a formulation may be prepared by uniformly and/or intimately bringing the active compound into association with a liquid carrier, a finely divided solid carrier, or both.

The amount of active agent or active agents administered can vary depending on various factors including, but not limited to, the weight, physical condition, and/or age of the subject; the particular clinical signs or symptoms exhibited by the subject; the type or cause of lung inflammation or pulmonary edema; and/or the method of administration. Thus, the absolute amount of active agent or active agents included in a given unit dosage form can vary widely, and depends upon factors such as the species, age, weight and physical condition of the subject, and/or the method of administration. Accordingly, it is not practical to set forth generally the amount that constitutes an amount of active agent or active agents effective for all possible applications. The physiologically active concentration of the active agent or active agents at the cellular level has been determined and varies depending upon the cell type and the specific hormonal target effect. Dosing of the active agent or active agents can be designed to achieve either physiologic or pharmacologic local tissue levels. Those of ordinary skill in the art, however, can determine the appropriate amount with due consideration of such factors.

For example, a D3 inhibitor, with or without other active agent may be administered to treat pulmonary edema or lung inflammation at the same dose and frequency for which the D3 inhibitor or another active agent has already received regulatory approval. For example, some inhibitors of D3 have received regulatory approval for treating other conditions, including, in some cases, hyperthyroidism. Approved deiodinase inhibitors include iopanoic acid (IOP), ipodate, propylthiourea (PTU), propylthiouracil, propranolol, dexamethasone, cortisol, a glucocorticoid, and amiodarone. In other cases, a D3 inhibitor and/or another active agent may be administered for treating alveolar edema or lung inflammation at the same dose and frequency at which the drug is being evaluated in clinical or preclinical studies. One can alter the dosages of the active agent and/or frequency at which the active agent or active agents are administered as needed to achieve a desired level of D3 inhibitor in the subject. Thus, one can use standard/known dosing regimens and/or customize dosing as needed.

For example, a combination therapy may be effective even if one or more components of the combination is administered at a dose or a frequency that is less than the component when administered alone. Thus, a combination therapy may provide desired efficacy while reducing the likelihood or severity of a side effect caused by a component of the combination composition.

As another example, the primary active form of T3—i.e., the form in which it has the greatest physiological activity—is when the T3 is “free”—e.g., not bound to large proteins such as albumin. The same concept may apply to certain T3 analogs and/or some T3 mimetics. Therefore, the physiologic effect of a given amount of a T3 analog or a T3 mimetic also may be influenced by the proteins and other aspects of the environment that it is introduced into. Thus, a smaller amount of a T3 analog or a T3 mimetic may be required to achieve an effective drug delivered dose for the methods described herein—i.e., in the “free” state and delivered directly to the nasosinus, intratracheal, intrabronchial, or alveolar airspace—than the dose of the T3 analog or T3 mimetic receiving regulatory approval for treating other conditions by, for example, intravenous delivery.

In some embodiments, the method can include administering sufficient active agent to provide a deposited dose of, for example, from about 0.5 ng to about 100 mg to the subject, although in some embodiments the methods may be performed by administering an active agent or active agents in a dose outside this range. In some of these embodiments, the method includes administering sufficient active agent or active agents to provide a deposited dose of from about 5 ng to about 50 μg to the subject. On a μg/kg basis, the calculated administered dose to achieve physiologic effects could range from as low as 2 ng/kg to 1 mg/kg. As one example, a 50 μg dose can provide a μg/kg dosage range of from about 0.03 μg/kg (to a 160 kg person) to as high as 25 μg/kg (to a 2 kg preterm infant). In many instances, however, dosing on a μg/kg basis is less relevant since direct instillation to lung tissue is not as subject to systemic dilution as, for example, intravenous administration. Lung size in adults does not vary significantly with weight, so mass of active agent or active agents delivered is often the more relevant measure of an appropriate dose.

As used herein, the term “deposited dose” or “lung-delivered” dose refers to the amount of active agent or active agents deposited to the surface of the respiratory tract. For instillation, the deposited dose is essentially the full dose being instilled. In an aerosol or nebulized formulation, however, the deposited dose is conventionally 10% or less of the drug being aerosolized or nebulized. 90% of the drug is expected to be lost in the delivery apparatus and/or exhaled. This may be greater in the injured ARDS lung. Thus, one may aerosolize or nebulize 500 μg of active agent or active agents to achieve an aerosolized or nebulized deposited dose of 50 μg. The use the term “deposited dose” or “lung-delivered” dose normalizes the dose across different routes of administration.

A sufficient deposited dose or lung-delivered dose can provide delivery of a minimum amount of active agent or active agents of at least 0.5 ng such as, for example, at least 1 ng, at least 5 ng, at least 5 ng, at least 10 ng, at least 20 ng, at least 50 ng, at least 100 ng, at least 1 μg, at least 10 μg, at least 50 μg, at least 100 μg, at least 250 μg, at least 500 μg, at least 1 mg, at least 1.5 mg, at least 2 mg, at least 5 mg, at least 10 mg, at least 15 mg, at least 20 mg, or at least 25 mg.

A sufficient deposited dose or lung-delivered dose can provide delivery of a maximum amount of active agent or active agents of no more than 100 mg such as, for example, no more than 50 mg, no more than 30 mg, no more than 20 mg, no more than 15 mg, no more than 10 mg, no more than 5 mg, no more than 4 mg, no more than 3 mg, no more than 2 mg, no more than 1.5 mg, no more than 1 mg, no more than 500 μg, no more than 300 μg, no more than 200 μg, no more than 100 μg, no more than 50 μg, no more than 30 μg, no more than 20 μg, or no more than 10 μg.

A sufficient deposited dose or lung-delivered dose also can be characterized by any range that includes, as endpoints, any combination of a minimum deposited dose or lung-delivered dose identified above and any maximum deposited dose or lung-delivered dose identified above that is greater than the minimum deposited dose or lung-delivered dose. For example, in some embodiments, the deposited dose or lung-delivered dose can be from 1 μg to 1.5 mg such as, for example, from 5 μg to 50 μg. A sufficient deposited dose or lung-delivered dose also can be equal to any minimum deposited dose or lung-delivered dose or any maximum deposited dose or lung-delivered dose. Thus, for example, a sufficient deposited dose or lung-delivered dose can be 1 ng, 100 ng, 1 μg, 5 μg, 50 μg, 1 mg, 1.5 mg, 10 mg, or 50 mg.

In some embodiments, an active agent or active agents may be administered, for example, from a single dose to multiple administrations per day, although in some embodiments the method can be performed by administering active agent or active agents at a frequency outside this range. When a dose is delivered via multiple administrations within a dosing period, the amount of each administration may be the same or different. For example, a dose of 50 μg in a day may be administered as a single administration of 50 μg, two 25 μg administrations, or in multiple unequal administrations. Also, when a dose is delivered via multiple administrations within a dosing period, the interval between administrations may be the same or be different. In certain embodiments, active agent or active agents may be administered from about once per day, four times per day, or continuously.

In some embodiments, an active agent or active agents may be administered, for example, from a single dose to a duration of multiple days, although in some embodiments the method can be performed by administering active agent or active agents for a period outside this range. In certain embodiments, an active agent or active agents may be administered once, may be administered over a period of three days, or may be administered over a period of seven days. In certain embodiments, an active agent or active agents may be administered from about once per day, four times per day, or continuously.

Treating alveolar edema, lung inflammation, or associated conditions can be prophylactic or, alternatively, can be initiated after the subject exhibits the onset of pulmonary edema or lung inflammation or the associated symptoms or clinical signs of a condition. Treatment that is prophylactic—e.g., initiated before a subject experiences an event (e.g., cancer radiotherapy) or manifests a symptom or clinical sign of the condition (e.g., while an infection remains subclinical)—is referred to herein as treatment of a subject that is “at risk” of having the condition. As used herein, the term “at risk” refers to a subject that may or may not actually possess the described risk. Thus, for example, a subject “at risk” of infectious condition is a subject present in an area where other individuals have been identified as having the infectious condition and/or is likely to be exposed to the infectious agent even if the subject has not yet manifested any detectable indication of infection by the microbe and regardless of whether the subject may harbor a subclinical amount of the microbe. As another example, a subject “at risk” of a non-infectious condition is a subject possessing one or more risk factors associated with the condition such as, for example, genetic predisposition, ancestry, age, sex, geographical location, or medical history. The subject may be an individual of any species susceptible to lung inflammation and/or pulmonary edema. Exemplary subjects include humans, non-human mammals (e.g., livestock animals, companion animals), birds, etc.

Accordingly, a pharmaceutical composition as described herein can be administered before, during, or after the subject first exhibits pulmonary edema, lung inflammation, or other symptom or clinical sign of associated conditions or, in the case of infectious conditions, before, during, or after the subject first comes in contact with the infectious agent. Treatment initiated before the subject first exhibits pulmonary edema or lung inflammation or another associated symptom or clinical sign may result in decreasing the likelihood that the subject experiences clinical consequences compared to a subject to whom the composition is not administered, decreasing the severity and/or completely resolving the lung abnormality. Treatment initiated after the subject first exhibits clinical manifestations may result in decreasing the severity and/or complete resolution of pulmonary edema and/or lung inflammation experienced by the subject compared to a subject to whom the composition is not administered.

For example, hyperoxic injury to rats in vivo and to alveolar type II cells in vitro is decreased when T3 levels are supported in advance of or coincident with injurious hyperoxic exposure. In vitro, alveolar type II cell death was significantly reduced. In vivo, lung inflammation, lung injury, neutrophil infiltration and protein leakage into the alveolar space were significantly reduced.

Thus, the method includes administering an effective amount of a pharmaceutical composition as described herein to a subject having, or at risk of having, pulmonary edema or lung inflammation. In this aspect, an “effective amount” is an amount effective to reduce, limit progression, ameliorate, or resolve, to any extent, the pulmonary edema or lung inflammation. For example, an “effective amount” of a pharmaceutical composition that includes D3 may increase alveolar fluid clearance, increase the population of alveolar type II pneumocytes, increase the size of alveolar type II pneumocytes, increase Na,K-ATPase activity in alveolar epithelial cells, decrease or repair alveolar damage, decrease hypoxemia, and/or decrease in inflammation throughout the respiratory tract (e.g., nasosinus, intratracheal, intrabronchial and alveolar airspace).

In the preceding description and following claims, the term “and/or” means one or all of the listed elements or a combination of any two or more of the listed elements; the terms “comprises,” “comprising,” and variations thereof are to be construed as open ended i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Human Lung Tissue Procurement

Post-mortem lung tissue was obtained from consecutive adult patients (male and female) with a clinical diagnosis of ARDS. Autopsies were authorized by the Institutional Review Board and performed after family consent, from December 2008 through October 2009. The diagnosis of ARDS was based on the following criteria: PₐO₂/FIO₂<200 mmHg, wedge <18 mmHg or CVP <12 mmHg, and CXR with bilateral patchy infiltrates as defined by the American European Consensus Conference. ARDS resulted from a variety of etiologies including pneumonia (viral or bacterial), sepsis, trauma and post-surgical lung injury (Table 1). Consecutive adult patients dying of non-pulmonary causes and undergoing autopsy by the Medical Examiner were used as controls (e.g., alcohol overdose, hypothermia, myocardial infarction, and motor vehicle trauma (Table 1).

TABLE 1
ARDS patients and normal controls

| | | Free T3 |
|---|---|---|
| ARDS Patients | ARDS Etiology | (pg/mL) |
| 23-year-old male | H1N1 influenza pneumonia | 1.2 |
| 44-year-old male | H1N1 influenza pneumonia | |
| 46-year-old female | MRSA pneumonia/sepsis | |
| 64-year-old male | PCP/CMV pneumonia | |
| 80-year-old male | Pneumococcal Pneumonia/sepsis | |
| 40-year-old male | Cholecystitis/sepsis | |
| 63-year-old female | Post-CABG surgery | 1.5 |
| 65-year-old female | Motor vehicle trauma | |
| | | Free T3 |
| Normal Controls | Cause of Death | (pg/mL) |
| 23-year-old male | Alcohol overdose | |
| 63-year-old male | Hypothermia | |
| 54-year-old male | Acute Myocardial infarction | 3.8 |
| 48-year-old female | Motor vehicle trauma | |

MRSA: Methicillin-resistant Staphylococcus aureus;
PCP: Pneumocystis carnii (jiroveci) pneumonia;
CMV: Cytomegalovirus;
CABG: Coronary artery bypass graft.

The lung samples were procured within four to twelve hours after death. Tissue samples were dissected from the peripheral/sub-pleural parenchyma of the anterior lung fields, sliced into 2-cm×2-cm pieces, flash frozen in liquid nitrogen, and stored at −80° C. for future assays or fixed in formalin and embedded for histological and immunochemical analysis. Staff pathologists (Department of Pathology and Laboratory Medicine, Essentia Health—St. Mary's Medical Center and Duluth Clinic, Duluth, Minn.) assigned a histologic diagnosis to each set of tissue. Lung samples demonstrating diffuse alveolar damage (DAD), including hypercellularity, and hyaline membrane/fibrin deposition, were used as study tissues. Lung samples from patients dying of non-pulmonary causes and demonstrating normal lung histologic architecture were used as control tissues. All samples with equivocal histology were excluded.

### Human Lung Deiodinase III (D3) Immunohistochemistry

Immunohistochemistry for detection of D3 was performed using a primary rabbit anti-deiodinase 3 antibody (1:100; gift of Domenico Salvatore, M. D., Ph.D., University of Naples Federico II, Naples, Italy), and a biotinylated goat anti rabbit secondary antibody followed by an avidin biotin complex (Vector Laboratories, Inc., Burlingame, Calif.). Diaminobenzidine (DAB) was used as the chromogen. The following protocol was used: Slides were deparaffinized in xylene and endogenous peroxidase activity was blocked with 0.3% hydrogen peroxide in methanol. The slides were rehydrated and treated with trypsin for 30 minutes at 90° C. After cooling, the sections were blocked with 10% normal goat serum in PBS+0.1% Tween-20 for 30 minutes. The anti-D3 antibody (1:100) was added for one hour at room temperature followed by washing in PBS and incubation with the secondary biotinylated goat anti-rabbit IgG antibody for 60 minutes at room temperature. Avidin Biotin Complex (Vector Laboratories, Inc., Burlingame, Calif.) was incubated with the tissue for 30 minutes followed by development of diaminobenzidine until the desired staining intensity was reached. The slides were counterstained for one minute with hematoxylin, dehydrated and examined. All tissue was identically processed with equal exposure time. Examination and photography was performed using a light microscope (DMRB, Leica Microsystems GmbH, Wetzlar, Germany).

### D3 Enzymatic Activity

Frozen lung tissue samples were thawed and sonicated in 0.1 M phosphate and 1 mM EDTA at pH 6.9 with 10 mM dithiothreitol and 0.25 M sucrose. D3 activity was assayed by HPLC using 150 μg of cellular protein, 200,000 cpm of 3, 5,[125I]3′-triiodothyronine (PerkinElmer, Inc., Waltham, Mass.), 1 mM 6N-propylthiouracil (PTU), 10 mM dithiothreitol (DTT), and 0-500 nM unlabeled T3 in each reaction as previously described (Simonides et al., J Clin Invest 118:975-983; 2008). Reactions were stopped by adding methanol and the products of deiodination were quantified by HPLC as previously described (Richard et al., J Clin Endocrinol Metab 83:2868-2874; 1998). D3 velocities were expressed as fmol of T3 inner-ring deiodinated per mg of sonicate protein per minute (fmol/mg/min). Samples with velocities below the detection limit of the assay were set to the minimum detectable activity (MDA) value, 0.05 fmol/mg/min. The MDA was calculated statistically as three standard deviations above background activity.

### Lung Total Tissue T3 Measurement

Thyroid hormones were extracted from human lung samples weighing ˜0.5 g using a modification of a previously-described method (Excobare et al., Endocrinology 117:1890-1900; 1985). Tissue was homogenized in 4 mL methanol containing 1 mM PTU (methanol-PTU) per gram tissue with a rotor-stator homogenizer at ˜30,000 rpm for 30 seconds. To assess individual sample percent recoveries, 100 μL of ¹²⁵I-T4 tracer (0.02 μg/L in methanol-PTU) was added to each sample. Chloroform was added at double the volume of methanol-PTU and samples were mixed by vortexing. The mixture was centrifuged at 2000 rpm for 15 minutes and the supernatant liquid was transferred to a clean 50 mL tube. The remaining pellets were subjected to two additional extractions by vortexing in 5 mL chloroform:methanol (2:1) per gram tissue, centrifuging at 2000 rpm for 15 minutes, and removing and combining the supernatant with the first extract. To the combined extracts, 1 mL 0.05% CaCl₂) was added for every 5 mL of extract. The mixture was vortexed and centrifuged at 2000 rpm for five minutes. The upper aqueous layer, containing thyroid hormones, was transferred to a clean 50-mL tube. The lower organic layer was re-extracted two more times with a volume of pure upper layer (chloroform:methanol:0.05% CaCl₂), 3:49:48) equal to the amount of upper layer removed in the previous step. The combined extracted upper layers were subjected to rotary evaporation to remove the remaining chloroform and methanol. The aqueous mixture was shell-frozen and evaporated to complete dryness by lyophilization. Each lyophilized sample was dissolved in 500 L stripped rat serum and T3 levels were measured using a serum total T3 RIA assay kit (Siemens Medical Solutions Diagnostics; Los Angeles, Calif.), as previously described (Bastian et al., Endocrinology 151:4055-4065; 2010).

### Statistical Analysis

Statistical analysis of D3 activities and tissue T3 levels was performed using one-way analysis of variance and Tukey's post hoc multiple comparison test. Statistical analyses and data graphing were carried out using the Prism (GraphPad Software, La Jolla, Calif.) software package.

Data are presented as mean±SEM. An α=0.05 was chosen to define significant differences.

### Example 2

### Study Design: First In-Human Clinical Trial as Described in FDA-Approved Investigative New Drug (#126204).

Informed consent is obtained within 24-hours prior to administering the study drug. For both the Treatment Group (Intervention) and Control Group (Non-Intervention), the study protocol will be started at Time 0 with a 6-hour EVLWI/PVPI measurement, a 12-hour EVLWI/PVPI measurement, a 24-hour EVLWI/PVPI measurement, a 48-hour EVLWI/PVPI measurement, a 72-hour EVLWI/PVPI measurement, and a 96-hour EVLWI/PVPI measurement.

Study Drug Human ARDS patients are treated with liothyronine sodium (T3), which is a synthetic form of thyroid hormone T3. Liothyronine sodium is provided in amber-glass vials containing 10 μg (10 mcg/ml in 1 ml vials) of liothyronine sodium in a sterile non-pyrogenic aqueous solution of 6.8% alcohol (by volume), 0.175 mg anhydrous citric acid, and 2.19 mg ammonium hydroxide. Prior to instillation, the liothyronine sodium is adjusted to neutral pH (6-8) by adding 1.0 N HCL prior to diluting in 0.9% normal saline (NS) under sterile conditions by an appropriately trained pharmacist.

Liothyronine sodium is formulated for administration as follows: 5 μg dose (0.5 ml liothyronine sodium+0.9% NS to 10 ml total volume); 10 μg dose (1.0 ml liothyronine sodium+0.9% NS to 10 ml total volume); 25 μg dose (2.5 ml liothyronine sodium+0.9% NS to 10 ml total volume); 50 μg dose (5.0 ml liothyronine sodium+0.9% NS to 10 ml total volume).

### Treatment Group (Intervention)

50 patients receive treatment. Upon enrollment and measurement of baseline values, patients receive 5 μg T3 by airway instillation. Patients are monitored for 24 hours for adverse effects and changes in EVLWI. After 24 hours, if no adverse effects are seen and EVLWI and/or PVPI is unchanged, patients receive a 2× escalated dose of 10 μg T3 by airway instillation. Patients are monitored for 24 hours for adverse effects and changes in EVLWI. and/or PVPI. At t=48 hours from first T3 dose, if no adverse effects are seen and EVLWI is unchanged, patients receive a 2.5× escalated dose of 25 μg T3 by airway instillation. Patients are monitored for 24 hours for adverse effects and changes in EVLWI and/or PVPI. At t=72 hours from first T3 dose, if no adverse effects are seen and EVLWI is unchanged, patients receive a 2× escalated dose of 50 μg T3 by airway instillation. A final EVLWI and PVPI measurement is made 24-hours after final T3 dose at time=96 hours (end time point).

### Control Group (Non-Intervention)

The control group includes 18 patients. Upon enrollment and measurement of baseline values, control patients receive no research intervention. Control subjects receive standard of care. EVLWI and PVPI are measured at Time 0 (before treatment), at six hours, 12 hours, 24 hours, 48 hours, 72 hours, and 96 hours.

### Primary Study Endpoints

Prior to commencing the study protocol and continuously thereafter, safety and tolerability of the airway to instilled T3 therapy are assessed. Subjects will be monitored for composite endpoints, including pulmonary events (e.g., progressive hemoptysis; quantity ≥30 ml blood-stained sputum), cardiac events (e.g., new sustained ventricular arrhythmia (duration >30 seconds); new sustained accelerated junctional arrhythmia (rate >80 bpm) with worsened hypotension; new sustained atrial fibrillation with rapid ventricular response (ventricular rate >160 bpm) with worsened hypotension; or cardiac arrest (asystole or pulseless electrical activity); and/or hypertensive crisis (systolic >200, or diastolic >120, or change in MAP >20 mmHg).

### Secondary Study Endpoints

To assess the efficacy of airway-instilled T3 on reducing EVLWI and/or PVPI in ARDS patients, EVLWI, PVPI, and oxygenation (arterial blood gas, ABG) are measured on subjects in both the Treatment Group and the Control Group beginning at baseline (T=0) and at six hours, 12 hours, 24 hours, 48 hours, 72 hours, and 96 hours thereafter. Additional serial measurements include blood pressure (BP), mean arterial pressure (MAP), central venous pressure (CVP), cardiac index (CI), systemic vascular resistance index (SVRI), oxygen saturation O₂ₛₐₜ, Finally, serum free T3, free T4, and TSH are measured at each time interval.

### Example 3

### Test System

This study was conducted using both male and female Sprague-Dawley rats (Envigo, Huntingdon, United Kingdom). Evaluation of the safety of the tracheal route of instillation for liothyronine sodium injection in human clinical trials can be accomplished in this species at appropriate dose levels. Furthermore, responses to thyroid hormone in rats are similar to responses in humans, and the choice of the rat model is based in large part on pharmacologic data from studies of thyroid hormone and associated receptors and physiological responses in rat lung. The University of Minnesota is accredited by the Association for the Assessment and Accreditation of Laboratory Animal Care, International (AAALAC) and registered with the United States Department of Agriculture to conduct research in laboratory animals. Animal studies conformed to NIH guidelines (Guide for the Care and Use of Laboratory Animals. NIH publication No. 86-23. Revised 1985). The protocol was reviewed and approved, as applicable, by the Institutional Animal Care and Use Committee (IACUC) at the University of Minnesota for compliance with regulations prior to study initiation or implementation of amended activities.

### Summary of Toxicology Study Design

Details of the study design are shown in Table 2. Sixty animals (plus two spare animals/sex/group) were anesthetized and dosed via intratracheal instillation of test or control materials for five consecutive days. On the day after the last dose a terminal blood collection was performed for clinical pathology, after which animals were euthanized and a gross examination of all organs was performed by a board certified veterinary pathologist. Select tissues were collected for histopathology. Twenty-four animals in the toxicokinetic (TK) phase were anesthetized and dosed with a single intratracheal instillation of T3. Terminal blood collection was performed at two designated time points per animal up to 24 hours after administration for toxicokinetic evaluation. TK animals were euthanized without further evaluation after the final blood collection. All animals were acclimated for a minimum of seven days prior to the dosing procedure. Animals underwent baseline observations and examinations prior to the initiation of the study, and clinical observations and body weight monitoring were performed throughout the in-life portion of the study. All animals received the same dose volume (0.3 mL) of either test or control materials. This maximum feasible dose (MFD) is constrained by the maximum volume that can be safely and reproducibly (over five days) instilled into lungs of rats weighing 250 g to 350 g, which was determined in preliminary toxicology studies to be 0.3 mL. Actual doses delivered are reported as both μg/kg body weight and μg/gm wet lung weight. The toxicity phase animals were 72-135 days of age at the time of initial dosing. Males weighed between 256.52 g and 307.50 g with a mean±standard deviation of 286.74±11.77 g, and females weighed between 250.46 g and 299.01 g with a mean±standard deviation of 260.64±10.34 g. The TK phase animals were 68-144 days of age at the time of initial dosing. Males weighed between 261.23 g and 316.19 g; females weighed between 250.06 g and 280.32 g.

TABLE 2
Study Design

| | | | Toxicokinetic | | |
|---|---|---|---|---|---|
| | Toxicity Phase | | (TK) Phase | | |
| | Number of | | Number of | | |
| | Toxicity Animals | | TK Animals | | Dose |
| Group | M | F | M | F | Volume |
| 1 T3 Vehicle | 10 + 2^{a} | 10 + 2^{a} | 0 | 0 | 300 μL |
| (Control) | | | | | |
| 2 Normal Saline | 10 + 2^{a} | 10 + 4^{a} | 0 | 0 | |
| (Control) | | | | | |
| 3 T3 (Test | 10 + 2^{a} | 10 + 2^{a} | 12 + 2^{b} | 12 + 2^{b} | |
| Article) | | | | | |
| Totals | 30 + 6 | 30 + 8 | 12 + 2 | 12 + 2 | |

| | | |
|---|---|---|
| | 60 + 14 | 24 + 4 |
| Dosing | *Once daily | Single Dose |
| Frequency & | for 5 days | |
| Duration | | |
| Scheduled | 1 day post | Day 1 |
| Termination | final dose | |
| TK Blood | NA | At designated |
| Collection | | time points |
| Histology | Yes | NA |

F = Females,
M = Males,
NA = Not Applicable
^{a}Use of Spares-Two spare animals of each sex/toxicity group were dosed with the animals from each group so that they were available for replacement within the similar timeframe. An additional two females were dosed in Group 2 due to early deaths experienced due to non-test material-related issues. The spare animals underwent terminal clinical pathology and gross necropsy evaluations.
^{b}Use of Spares-Four (4) additional unused spares were released from study at the direction of the Study Director.

### Test and Control Materials

T3 used for this study was liothyronine sodium injection (X-GEN Pharmaceutical S, Inc., Horseheads, N.Y.) supplied in 1.0 mL amber glass vials at a concentration of 10 μg in 1.0 mL. Each mL of liothyronine sodium injection contains, in sterile, non-pyrogenic USP grade water, liothyronine sodium equivalent to 10 μg of liothyronine (T3), 6.8% alcohol by volume, 0.175 mg anhydrous citric acid and 2.19 mg ammonia (as ammonium hydroxide). In preliminary studies it was determined that rats do not tolerate intratracheal instillation of liothyronine sodium at a pH of >10.0 as it is supplied commercially. Therefore, the liothyronine sodium and the vehicle were adjusted to neutral pH (6.0-8.0) with sterile 1.0 N HCl (Sigma-Aldrich, St. Louis, Mo.) added aseptically in a biosafety cabinet prior to intratracheal instillation into animals. Using aseptic technique, vials of liothyronine sodium were opened and the entire contents were transferred into sterile 1.5 mL Eppendorf tubes. 80-90 μL of 1.0 N HCl was added to the tube and gently vortexed, and the pH was measured using pH test strips (pH 4.5 to 10.0, Ricca Chemical Co., Arlington, Tex.). Additional HCl was titrated in gradually, as needed, until the pH was in the desired range. There is a volume increase of approximately 10% after adjusting the pH to neutral with 1.0 N HCl, resulting in a final concentration of liothyronine sodium of approximately 2.73 μg T3/300 μL (9.17 μg/ml). This solution was stored at 4° C. and used for up to 26 hours after pH adjustment procedure. The vehicle control solution was prepared in sterile, non-pyrogenic USP grade water (USP/EP Purified, Ricca Chemical Co., Arlington, Tex.) and contained, per mL of solution, 6.8% ethanol (Decon Laboratories, Inc., King of Prussia, Pa.) by volume, 0.175 mg anhydrous citric acid (Sigma-Aldrich, St. Louis, Mo.) and 2.19 mg ammonia (J. T. Baker Chemical Co., Avantor Performance Materials LLC, Radnor, Pa.), ammonia solution, strong 27.0-30.0%, N.F.-F.C.C.). The vehicle was also adjusted to neutral pH (6.0-8.0) with 1.0 N HCl as described above. Using aseptic technique the vehicle was filter sterilized and aliquoted into 21 sterile disposable tubes (5 mL each) and stored at 4° C., and a new tube was opened for each day of use. Normal Saline (0.9% Sodium Chloride Injection USP, B. Braun Medical, Inc., Bethlehem, Pa.), was stored at room temperature. A new package was opened for each day of use.

### In-Life Animal Care

Upon arrival animals were visually examined by trained staff and weighed, counted, sexed, and appropriately separated into housing boxes. Each animal received a metal ear tag containing an individual identifier prior to initial dosing. Animals were housed in AAALAC accredited pens under sanitary conditions and were socially housed to provide enrichment and companionship. The temperature and humidity of the housing area was monitored a minimum of once daily. Animals were acclimated for a minimum of seven days prior to dosing initiation. Preconditioning was allowed during this period to acclimate the animals to the handling they would experience during weighing, examinations and dosing procedures. All animals were given food (TEKLAD, Envigo, Huntingdon, United Kingdom) and potable tap water ad libitum. Animals were not fasted for procedures. Veterinary care was available throughout the course of the study. Observations on general health, including animal activity, appearance, food and water intake, mortality/moribundity and other endpoints (Table 3) were performed and recorded at least once daily from the time of enrollment on study until euthanasia by a trained technician. A Veterinarian was notified of abnormalities in activity or appearance. To prevent bias with regard to observations, health concerns or treatments, veterinary and general animal care personnel were not informed of dose group distribution.

TABLE 3
In-life Observations/Assessments & Health Monitoring

| Activity | Frequency |
|---|---|
| Handling & Restraint | SID beginning within 2 days after arrival |
| Pre-Conditioning | |
| Body Weight (grams) | Within 1 days prior to and/or on the day |
| | of initial dose. |
| | Minimally 2-3 times/week during dosing |
| | Day of term |
| Cage Side Observations | Minimally SID beginning within 3 days |
| Mortality/Alive or Dead | prior to initial dose. |
| Defecation | Day of term |
| Urination | |
| Behavior/vocalization | |
| Posture/Attitude/Activity | |
| Food available | |
| Water available, bottle intact | |
| Bedding sufficient | |
| Detailed Clinical Observations | At least once prior to initial dosing |
| Posture/Attitude | At least once after initial dose |
| Integument/Hair | Day of Term |
| Eyes | |
| Ears | |
| Nose | |
| Respiratory | |
| Musculoskeletal | |
| Urogenital | |
| Gastrointestinal | |
| Neurological | |

### Dosing Procedure

Test and control materials were drawn into dosing syringes using aseptic technique. Using a 18G needle, 0.5 mL of air was drawn into a 1 cc syringe followed by 0.3 mL (300 μL) of the solutions. Animals were anesthetized with a combination of ketamine, 40 mg/kg to 200 mg/kg, and xylazine, 1 mg/kg to 7 mg/kg intraperitoneally (TP), to effect. The dose was adjusted daily, as needed, based on individual animal response and recovery. Depth of anesthesia was evaluated by toe pinch, and eye lubricant was applied to the eyes. An upright, inclined stand was used to support the animals in the desired position during the dosing procedure by suspending the animals from a soft, non-latex rubber band at the top of the stand by their front incisors. Up to 20 μL of 2% lidocaine was applied topically to the back of the throat using a blunt gavage needle prior to intubation with a tracheal catheter to minimize laryngeal spasms and facilitate tracheal placement. The animals were removed from the stand and positioned in prone position while the lidocaine took effect.

After allowing adequate time for lidocaine to take effect, the animals were again suspended on the apparatus, and a catheter (INTRAMEDIC 1.19 mm inner diameter, 1.70 mm outer diameter, Thermo Fisher Scientific, Waltham, Mass.) was inserted into the trachea by first visualizing the larynx through the oral cavity with the aid of an external light source directed at the throat. Holding the tongue aside with blunt forceps and gauze moistened with water helped with visualization of the airway. The catheter was advanced into the trachea to a pre-determined depth approximately 1.0 cm short of the branch point of the major bronchi (measured on a cadaver animal with the trachea and bronchi exposed). Catheter placement in the airway was verified by the fogging of a dental mirror placed at the opening of the catheter. The needle on the dosing syringe was then inserted into the catheter, and the test material and bolus of air was rapidly delivered in a one-to-two second interval. The air bolus administered after the test material facilitated administration of the fluid into the lower airways and ensured that fluid was not retained in the trachea or major bronchi, as confirmed in preliminary experiments using a dye solution. The tracheal catheter was removed from the airway and the animal gently removed from the support apparatus. The animal was placed in a prone position on a heating pad with the chest elevated for a minimum of two minutes after instillation. After two minutes, the animal was placed flat on a heating pad until fully recovered.

### Terminal Euthanasia Procedures

### Blood Collection for Clinical Pathology (Hematology and Clinical Chemistry)

Blood samples from the toxicity phase animals for clinical pathology were collected one day after the final (fifth) intratracheal dose. Animals were anesthetized with isoflurane 2-5% and oxygen 1-1.5 L/min by inhalation anesthesia via nose cone as needed. For hematology, ≥0.5 mL whole blood was collected via the orbital sinus through plain or coated microhematocrit capillary tubes into K₂EDTA collection tubes (BD Biosciences, Thermo Fisher Scientific, Waltham, Mass.) containing an additional 30 μL of 2% EDTA solution (Sigma-Aldrich, St. Louis, Mo.), and kept at 4° C. until same day analysis. For serum chemistry, ≥0.75 mL whole blood was collected via the orbital sinus through uncoated capillary tubes into red top serum microtubes (Sarstedt AG & Co. KG, Numbrecht, Germany). For serum collection, tubes were maintained at room temperature for 30 to 60 minutes after collection and then centrifuged at 10,000×g for five minutes at 4° C. The resultant serum was separated and stored at ≤−70° C. if analysis was to occur the following day or kept at 4° C. for same day analysis. All samples were sent to the University of Minnesota-Veterinary Medical Center (VMC) clinical pathology laboratory for analysis. Parameters evaluated for hematology are provided in Table 4. Parameters evaluated for clinical chemistry are provided in Table 5. Following blood collections animals were euthanized with EUTHASOL (Virbac Corp., Fort Worth, Tex.) ≥86 mg/kg IP to effect prior to necropsy. Assessment of the clinical pathology values was performed by Jill Schappa Faustich, DVM, DACVP, University of Minnesota.

TABLE 4
Hematology

Red Blood Cell Count (RBC)
Hemoglobin Concentration (HGB)
Free Plasma HGB
Hematocrit (HCT)
Mean Corpuscular Volume (MCV)
Mean Corpuscular Hemoglobin (MCH)
Mean Corpuscular Hemoglobin Concentration (MCHC)
Reticulocyte Count (Retic Absolute And Relative)
Platelet Count (Plt & PCT Absolute And Relative)
Mean Platelet Volume (MPV)
Platelet Distribution Width (PDW)
Red Cell Distribution Width (RDW)
White Blood Cell Count (WBC)
Neutrophil Seg Count (NEUT Absolute And Relative)
Neutrophil Band Count (BAND Absolute And Relative)
Lymphocyte Count (LYMPH Absolute And Relative)
Monocyte Count (MONO Absolute And Relative)
Eosinophil Count (EOS Absolute And Relative)
Basophil Count (BASO Absolute And Relative)
Mast Cells (Absolute And Relative)
Unclassified Cell Count (Absolute And Relative)

TABLE 5
Serum Chemistry

Blood Urea Nitrogen (BUN)
Creatinine (Creat)
Calcium (Ca)
Phosphorous (Phos)
Magnesium (Mg)
Protein (TP)
Albumin (Alb)
Globulin (Glob)
Alb/Glob ratio^{a}
Sodium (Na)
Chloride (Cl)
Potassium (K)
Bicarbonate (HCO3)
Osmolality (Osmol)
Anion Gap (An Gap)
Bilirubin, Total (T.Bili)
Alkaline Phosphatase (ALP)
Gamma-Glutamyl Transferase(GGT)
Alanine Transferase (ALT)
Aspartate Transferase (AST)
Creatine Kinase (CK)
Glucose (Gluc)
Cholesterol (Chol)
Amylase
Lipemia Icterus Hemolysis (LIH)

For toxicokinetic experiments, rats were anesthetized with combination of ketamine 40 mg/kg to 200 mg/kg and xylazine 1 mg/kg to 7 mg/kg, intraperitoneally (IP), to effect for dosing procedures, and dosed intratracheally with liothyronine sodium injection as previously described. The details of the TK sample collection protocol are provided in Table 6 and Table 7. Depending on the duration of time between dosing and the first or second blood collection time points, animals either had blood collected while still anesthetized under the injectable anesthetics, or if recovered, they were anesthetized with Isoflurane 2-5% and oxygen 1-1.5 L/minute by inhalation anesthesia via nose cone, as needed, to maintain adequate anesthesia depth (assessed by toe pinch). Topical proparicaine anesthetic ophthalmic solution was applied to each eye prior to performing the first blood collection and allowed time to take effect. Collection of serum samples for TK analysis was as described for serum chemistry samples above, and samples were stored at ≤−70° C. until assayed. Animals were euthanized with EUTHASOL (Virbac Corp., Fort Worth, Tex.) ≥86 mg/kg IP to effect following the final blood collection.

TABLE 6
Toxicokinetic Blood Collections

| | | Sample Collection Time Points | | | | | | | |
|---|---|---|---|---|---|---|---|---|---|
| | | Number of animals M/F per Occasion (Target | | | | | | | |
| | Total | Time Post Dose) Two time points per animal | | | | | | | |
| TK | Number of | | 15 | 30 | 1 | 2 | 4 | 6 | 24 |
| Group | Males/Females | T0 | min | min | hr | hr | hr | hr | hr |
| Test | 12/12 | 3/3 | 3/3 | 3/3 | 3/3 | 3/3 | 3/3 | 3/3 | 3/3 |
| Article | | | | | | | | | |

TABLE 7
Toxicokinetic Animal Distribution per Timepoint

| | T0 | 15 min | 30 min | 1 hr | 2 hr | 4 hr | 6 hr | 24 hr |
|---|---|---|---|---|---|---|---|---|
| Male | X | | X | | | | | |
| Male | | X | | | X | | | |
| Male | | | | X | | | | X |
| Male | | | | | | X | X | |
| Female | X | | | | X | | | |
| Female | | X | X | | | | | |
| Female | | | | X | | | | X |
| Female | | | | | | X | X | |

Three animals of each sex/timepoint

### Bioanalytical Procedure

For assessment of serum T3 levels samples were sent to the Fairview University of Minnesota Medical Center East Bank Diagnostic Laboratory for analysis, a clinical laboratory certified by CLIA and CAP. Prior to sending serum samples to the analytical lab each sample was diluted 1:4 or 1:8 in normal (0.9%) saline. These dilutions, determined in preliminary studies, ensured that sample total T3 concentrations would fall within assay range (10 μg/mL to 460 μg/mL). Samples were analyzed by a chemiluminescence assay for total triiodothyronine (T3).

### TK Analysis

An evaluation of the TK analysis was performed by Dick Brundage, PhD, University of Minnesota. Toxicokinetic parameters were estimated using Phoenix 64 WinNonlin pharmacokinetic software version 7.0. (Pharsight Corp., Mountain View, Calif.). A non-compartmental (NCA) approach consistent with the route of administration was used for parameter estimation. All parameters (Table 8) were generated from mean T3 concentrations in serum from all timepoints unless otherwise stated. Whenever possible, mean concentrations were derived from three animals/gender/time point. Parameters were estimated using sampling times relative to the start of each dose administration. The raw data was converted to ng/ml of serum by dividing the μg/dl values by 100 and then multiplying by the dilution factor for that sample, either 4 or 8. Values below the limit of quantification were calculated as 0.

TABLE 8
TK Parameters Estimated

| Parameter | Description of parameter |
|---|---|
| Cₘₐₓ | The maximum observed arithmetic mean |
| | concentration of T₃ measured after dosing. |
| Cₘₐₓ/D | The Cₘₐₓ divided by the dose administered. |
| Tₘₐₓ | The time after dosing at which the |
| | maximum observed arithmetic mean |
| | concentration of T₃ was observed. |
| AUC₍₀₋ₜ₎ | The area under the T₃ arithmetic |
| | mean concentration versus time curve from |
| | time zero the time after dosing at which the |
| | last quantifiable concentration of the drug |
| | was observed estimated by the linear or |
| | linear/log trapezoidal method. |
| AUC₍₀₋ₜ₎/D | The AUC₍₀₋ₜ₎ divided by the dose |
| | administered. |

When data permitted, the slope of the terminal elimination phase of
each arithmetic mean concentration versus time curve was determined
by log-linear regression, and the following additional parameters
were estimated:

Additional Parameters Estimated

| Parameter | Description of parameter |
|---|---|
| T½ | The apparent terminal elimination half-life. |
| AUC_{(0-inf)} | The area under the arithmetic mean |
| | concentration versus time curve from time |
| | zero to infinity. |
| AUC_{(0-inf)}/D | AUC_{(0-inf)} divided by the dose administered. |
| CL | Clearance: the apparent volume of plasma |
| | cleared of T₃ per unit time following |
| | intravenous dosing. |
| V_{d} | The apparent volume of distribution of T₃, |
| | determined from the terminal elimination |
| | phase following intravenous dosing. |

Calculation of arithmetic means and standard deviations for the matrix concentration data was performed/replicated in EXCEL (Microsoft, Corp., Redmond, Wash.) for reporting purposes. In addition to parameter estimates from mean concentration vs. time curves, the standard error of the AUC₍₀₋ₜ₎ and Cₘₐₓ by dose group, day, and gender (as appropriate) were generated using WINNONLIN (Cetara USA, Inc., Princeton, N.J.).

Cₘₐₓ and Tₘₐₓ were obtained by inspection of the data. Since measurable endogenous compound is present based on the observed concentration at time zero, a baseline subtraction was performed. Using the mean concentration data, the concentration at time zero was subtracted from the remaining concentrations for male and female animals. The area-under-the-curve (AUC) of the baseline subtracted concentrations was calculated using the linear trapezoidal rule. Since the 24-hour concentration in both male and female animals had approximately returned to the baseline (pre-dose) concentration, these observations were ignored in calculations for the AUC and half-life. The terminal elimination half-life was calculated from the last three observations at 2 hours, 4 hours, and 6 hours. WinNonlin NCA performs linear regression on the logs of the concentrations. The Uniform weighting scheme was selected. The default regression algorithm for NCA will not use Cₘₐₓ in the calculation of half-life, even if it appears to be part of the log-linear profile, nor will it provide any half-life based on only two observations. The default regression for the male animals was used. However, for the female animals, the concentration at time two hours was also the Cₘₐₓ value. Since it appeared to fall on the regression line of all three concentrations (adjusted R squared=1.0), it was included in the calculation of the half-life. Parameters were evaluated as appropriate at the discretion of the evaluator. Results are provided as individual values, and include graphing of mean and standard error using EXCEL (Microsoft, Corp., Redmond, Wash.) and WINNONLIN (Cetara USA, Inc., Princeton, N.J.) per appropriate groups when possible.

### Necropsy Procedures

### Gross Pathology

Toxicity Phase animals that were euthanized at scheduled termination or that were found dead or euthanized prior to scheduled termination, were subjected to an extensive necropsy performed by a board certified veterinary pathologist. The necropsy included an examination of the animal carcass and musculoskeletal system, external surfaces and all of its orifices, and cervical, thoracic, abdominal and pelvic regions, cavities and contents. Eyes were not examined due to terminal orbital blood collection methods.

### Histopathology

The primary target tissues assessed in this study for histopathalogic changes included the lungs, the trachea-bronchi branch point and the tracheobronchial lymph nodes. The intact heart-lung pluck including all target tissues noted above was removed from the animal intact. The heart-lung pluck was weighed, photographed and the lungs were then perfusion inflated via the trachea with 10% neutral buffered formalin (NBF). For inflation, an 18 g butterfly catheter connected to a reservoir of 10% NBF was inserted into the trachea and the lungs inflated for two minutes at a constant pressure of ˜20-25 cm, after which the trachea was tied off with suture to maintain inflation of the lungs during fixation. The entire heart lung pluck was then immersion fixed in 10% NBF. Prior to further processing for histology, the heart, trachea and any other adherent tissues were removed from the lungs and weighed. This weight, when subtracted from the weight of the heart-lung pluck taken at necropsy, provided the wet lung weight used in subsequent calculations of actual dose delivered. Non-target tissues including the brain, heart, liver, spleen, pancreas, kidneys and adrenal glands were evaluated for gross lesions. The non-target organs were collected whole with the exception of the liver, in which a representative specimen was collected from the anterior right lobe, and were stored in 10% NBF for potential future analysis. Histological processing and evaluations were performed by Dr. Joan Wicks, DVM, PhD, DACVP, Alizée Pathology, LLC, Thurmont, Md.

### Dose Administration

All doses were administered via intratracheal instillation at the maximum volume that could be safely and reproducibly delivered daily for five consecutive days, determined in preliminary studies to be 0.3 mL (300 μl) for rats weighing 250-350 grams. There were no apparent complications with the administration of the materials with the exception of one instance of 20-50 μl of vehicle control article coming out of the top of the dosing syringe during Dose 3 administration to LRT 633 (T3 vehicle group).

The calculated dose of T3 administered based on body weight on the initial day of administration (Day 1) and based on calculated wet lung weights are detailed in Table 9.

TABLE 9
Calculated T3 Dose. Toxicity Phase

| | | | Group 3 | Group 3 |
|---|---|---|---|---|
| Calculated Dose | | Group 3 All | Males | Females |
| μg T3/g Wet Lung | Average | 1.57 (0.61) | 1.50 (0.15) | 1.63 (0.14) |
| Weight | (SD) | | | |
| μg T3/kg Body | Average | 10.00 (0.64) | 9.45 (0.33) | 10.55 (0.25) |
| Weight | (SD) | | | |

1.0 ml T3 (10 μg/ml) diluted with ~100 μl 1.0N HCl to pH, 10 μg in 1.10 ml = 2.73 μg in 300 μl dose

### Toxicokinetics (TK)

Liothyronine sodium (T3) was successfully quantified for all of the samples submitted. All reported values were within the limits of quantification for the assay (10 μg/mL-460 μg/mL).

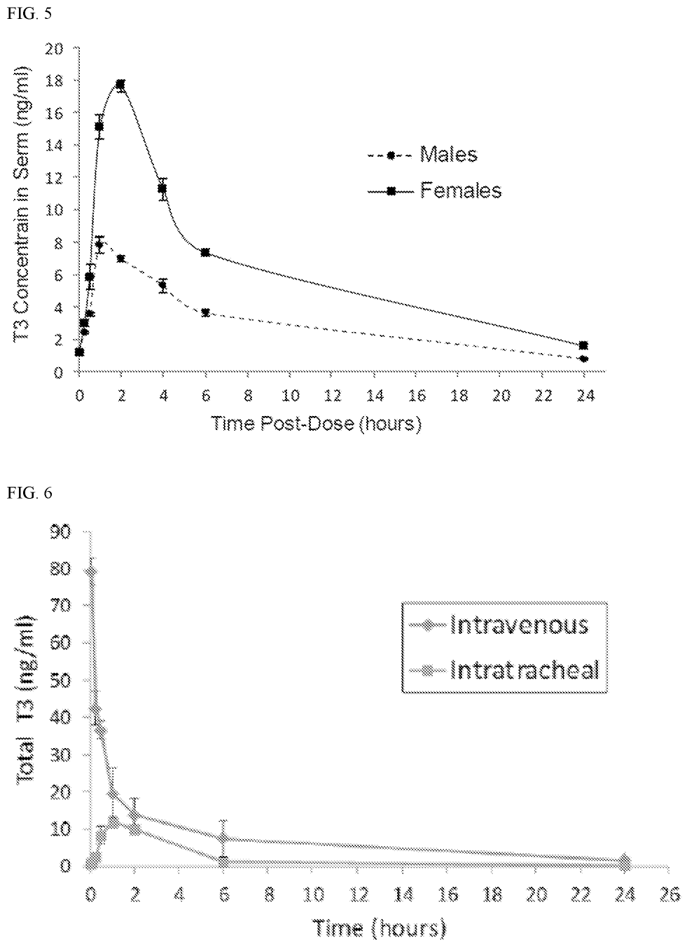

The measurable values are listed in Table 10 and are graphed in FIG. 5 (mean±standard error). TK analysis was performed on diluted samples from all 24 animals that received a single T3 dose (Table 11).

TABLE 10
T3 Detected in Serum in Single Dose TK Study
T₃ (ng/mL) Means and Standard Errors of Mean (SEM)

| | 0 | 15 min | 30 min | 1 hr | 2 hr | 4 hr | 6 hr | 24 hr |
|---|---|---|---|---|---|---|---|---|
| Male | 1.16 | 2.24 | 3.48 | 8.44 | 6.72 | 6.08 | 3.96 | 0.76 |
| | 1.28 | 2.52 | 3.76 | 6.84 | 6.92 | 4.52 | 3.24 | 0.80 |
| | 1.20 | 2.44 | 3.36 | 8.16 | 7.28 | 5.36 | 3.64 | 0.72 |
| Mean | 1.21 | 2.40 | 3.53 | 7.81 | 6.97 | 5.32 | 3.61 | 0.76 |
| SEM | 0.04 | 0.08 | 0.12 | 0.49 | 0.16 | 0.45 | 0.21 | 0.02 |
| Female | 1.44 | 2.56 | 4.92 | 15.60 | 17.68 | 12.32 | 7.52 | 1.84 |
| | 0.88 | 3.20 | 5.16 | 16.08 | 18.32 | 11.48 | 6.92 | 1.24 |
| | 1.04 | 3.24 | 7.44 | 13.68 | 16.96 | 10.00 | 7.64 | 1.60 |
| Mean | 1.12 | 3.00 | 5.84 | 15.12 | 17.65 | 11.27 | 7.36 | 1.56 |
| SEM | 0.17 | 0.22 | 0.80 | 0.73 | 0.39 | 0.68 | 0.22 | 0.17 |

TABLE 11
Noncompartmental analysis of TK samples

| | Cmax | Cmax_D | Tmax | HL_Lambda_z | AUClast |
|---|---|---|---|---|---|
| Sex | (ng/mL) | (ng/mL/ug) | (hr) | (hr) | (hr*ng/mL) |
| F | 16.53 | 6.12 | 2.00 | 2.85 | 64.08 |
| M | 6.60 | 2.44 | 1.00 | 3.17 | 25.38 |

| | AUCINF_obs | AUCINF_D_obs | Cl_F_obs | Vz_F_obs |
|---|---|---|---|---|
| Sex | (hr*ng/mL) | (hr*ng/mL/ug) | (mL/hr) | (mL) |
| F | 89.70 | 33.22 | 30.10 | 123.60 |
| M | 36.34 | 13.46 | 74.29 | 339.44 |

### Example 4

### Cell Culture and Hyperoxia Exposure

The adult rat AT2 cell line RLE-6TN (ATCC, Manassas, Va.) was cultured in DMEM/F12 medium with 10% FBS and in a 95% air, 5% CO₂ environment until they reached ˜50% confluence, then the cells were exposed to 95% O₂, 5% CO₂ in the presence or absence of T3 in DMEM/F12 with 2% stripped FBS for specified time periods. At the end of the hyperoxia exposure period, the viable cells were counted by trypan blue dye exclusion.

### Nuclear Extraction

Nuclei were extracted with NE-PER nuclear and cytoplasmic extraction reagents kit (Thermo Fisher Scientific, Inc., Waltham, Mass.) following the manufacturer's instruction.

### Cell Lysis and Western Blot

The cells were lysed in lysis buffer containing 20 mM Tris HCl (pH 7.5), 150 mM NaCl, 1 mM EDTA, 1 mM EGTA, 1% (vol/vol) Triton X-100 with protease inhibitors (1 mM PMSF, 2 μg/ml pepstatin, and 10 μg/ml each of aprotinin and leupeptin), and phosphatase inhibitors (2.5 mM sodium pyrophosphate, 1 mM β-glycerophosphate, and 1 mM Na₃VO₄). The lysate was drawn 10 times through a 25-gauge needle on ice for further lysis and then was centrifuged at 13,000 rpm for 15 minutes at 4° C. The supernatant was collected, and the protein concentrations were determined by use of the BCA protein assay kit (Sigma-Aldrich, St. Louis, Mo.). Immediately after this step, equal amounts of protein were subjected to Western blotting analysis.

### Nuclear Extraction.

Nuclei were extracted with NE-PER nuclear and cytoplasmic extraction reagents kit (Thermo Fisher Scientific, Inc., Waltham, Mass.) following the manufacturer's instruction.

Statistics All data are expressed as means±SD of a minimum of three or more independent experiments, unless otherwise noted. In most experiments, individual data points within an experiment represent the mean of at least two replicates. Comparisons involving three or more groups were analyzed by ANOVA and post hoc pairwise comparisons. Differences between means were considered significant at P<0.05.

### Example 5

### Experimental Design

All experimental protocols for animal treatments were approved by the University of Minnesota Institutional Animal Care and Use Committee. Specific pathogen-free (SPF) adult male Sprague Dawley rats (250 g-300 g) receiving intraperitoneal (ip) injections of either saline or T3 were exposed to normobaric hyperoxia (FI_{O2}>95%, 5 LPM) in a chamber with ad libitum access to food and water at room temperature for 48 or 60 hours to induce lung inflammation and injury. The room-air control rats were kept in the University animal housing facility. Rats were injected intraperitoneally with T3 or saline at doses and time points detailed in two protocols summarized in Table 12. At the end of hyperoxic exposure, rats were sacrificed by intraperitoneal pentobarbital injection, and the lungs were harvested; the right lobes were allocated for histopathology, measurement of lung tissue myeloperoxidase (MPO) activity and wet-to-dry weight ratio. The left lobes underwent bronchoalveolar lavage (BAL) to determine BAL protein concentrations and differential cell counts.

TABLE 12
Experimental protocols

| | 0 | 12 | 24 | 36 | 48 | 60 |
|---|---|---|---|---|---|---|
| | hours | hours | hours | hours | hours | hours |
| Protocol 1 | | | | | | |
| Control (saline) | ip | ip | ip | ip | ip | end |
| T3 (12.5 μg/kg) | ip | ip | ip | ip | ip | end |
| Protocol 2 | | | | | | |
| Control (saline) | ip | | ip | | end | |
| T3 (15.0 μg/kg) | ip | | ip | | end | |

### Wet-Dry Lung Weight Ratios

A portion of the right lung was rinsed briefly in PBS, blotted, and then weighed to obtain the “wet” weight. Lungs then were dried in an oven at 80° C. for seven days to obtain the “dry” weight.

### Lung Lavage Analyses

Bronchoalveolar lavage (BAL) of the left lung was performed using a modification of a method previously described (Pace et al., Exp Lung Res 35:380-398, 2009). Briefly, 4 mls of ice-chilled 1×PBS (pH 7.4) were instilled into the left lung, withdrawn, and re-instilled two subsequent times prior to analysis of the lavage fluid. The retrieved BAL fluid was centrifuged at 1500 rpm for 10 minutes to remove cells and debris. The cell pellet was resuspended in 1 ml of 1×PBS (pH 7.4) and total cell number was counted using a hemocytometer. BAL cytospin preparations were stained using the Hema3 stain kit (Thermo Fisher Scientific, Inc., Waltham, Mass.) to identify the nucleated cells. The protein concentration was determined on the supernatants of BAL fluid using a standard BCA assay (Sigma-Aldrich, St. Louis, Mo.).

### Myeloperoxidase (MPO) Assay

To quantify the neutrophil activity in the lung, MPO activity was assayed as previously described (Abraham et al., J Immunol 165:2950-2954, 2000). Lung tissues without prior lavage were frozen in liquid nitrogen, weighed, and stored at −86° C. The lungs were homogenized for 30 seconds in 1.5 ml 20 mM potassium phosphate, pH 7.4, and centrifuged at 4° C. for 30 minutes at 40,000×g. The pellet was resuspended in 1.5 ml 50 mM potassium phosphate, pH 6.0, containing 0.5% hexadecyltrimethylammonium bromide, sonicated for 90 seconds, incubated at 60° C. for two hours, and centrifuged at 14,000 rpm for 30 minutes at 4° C. The supernatant was assayed for peroxidase activity corrected to lung weight. MPO was expressed as activity per gram of lung tissue.

### Histochemistry

Lung tissue was removed and inflation fixed at 20 cm water pressure in 4% paraformaldehyde, paraffin embedded, cut as 5 micron sections and mounted onto poly-L-lysine slides. Sections were deparaffinized in xylene, rehydrated through a graded alcohol series in methanol, and placed in a 98° C. water bath for 30 minutes in citrate buffer (pH 6.0) for antigen retrieval. After quenching with 0.3% hydrogen peroxide in PBS, sections were incubated in normal serum for 30 minutes and for 15 minutes each with Avidin/Biotin Blocking Kit (Vector Laboratories, Inc., Burlingame, Calif.). After overnight incubation with Myeloperoxidase Ab-1 (Thermo Fisher Scientific, Inc., Fremont, Calif.) at 4° C., Biotinylated goat anti-rabbit IgG (1:500) and RTU Streptavidin (Vector Laboratories, Inc., Burlingame, Calif.) were applied sequentially for 30 minutes and 3,3′-diaminobenzidine was used as a peroxidase substrate. Sections were counterstained with hematoxylin. Image analysis and photography used a Leica Leitz DMRB microscope.

### Serum T3 Measurements

Blood samples were collected at the end of 60 hours of hyperoxia and were centrifuged at 13,000 rpm for 30 minutes at 4° C. Supernatant was stored at −20° C. Serum total T3 concentrations were measured with commercial RIA kits (Siemens Medical Solutions Diagnostics, Los Angeles, Calif.) as previously described (Bastian et al., Endocrinology 151:4055-4065, 2010).

### Statistical Analysis

Values were expressed as means±SD of a minimum of three experiments. Comparisons involving three or more groups were analyzed by ANOVA and post hoc pairwise comparisons. Differences between means were considered significant at p<0.05, adjusted for the number of comparisons.

The complete disclosure of all patents, patent applications, and publications, and electronically available material cited herein are incorporated by reference in their entirety. In the event that any inconsistency exists between the disclosure of the present application and the disclosure of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A pharmaceutical composition for administering directly to the lung of a subject, the compositions comprising:
an active agent effective to increase T3 concentration in the lung of the subject, the active agent comprising a deiodinase inhibitor, a thyroid hormone mimetic, or a thyroid hormone analog;
and a pharmaceutically acceptable buffer, adjusted to a pH of 5.5-8.5.

2. The pharmaceutical composition of claim 1, wherein the active agent effective to increase T3 concentration in the lung of the subject comprises iopanoic acid, iopanoate, ipodate, propylthiourea, propylthiouracil, 6-propylthiouracil, propranolol, D-propranolol, dexamethasone, cortisol, a glucocorticoid, amiodarone, desethlaminodarone, dronedarone, (3′),4′,4,6-(tetra)trihydroxyaurone, insulin, 3′,5′-cyclic adenosine monophosphate, butyrate, a phenolphthalein dye, or an environmental halogenated chemical.

3. The pharmaceutical composition of claim 1, wherein the active agent effective to increase T3 concentration in the lung of the subject comprises a thyroid hormone derivative, a thyronine, a thyronamine, a thyroacetic acid, a chemically-modified thyroid hormone, a thyroid-hormone-linked macromolecule, or a thyroid hormone receptor agonist.

4. The pharmaceutical composition of claim 3, wherein the active agent effective to increase T3 concentration in the lung of the subject comprises thyroxine; 3,3′,5-triiodothyronine; 3,5-dimethyl-3′-isoprophylthyronine; 3,5-dibromo-3′-pyridazinone-L-thyronine; 3,3′,5,5′ tetraiodoacetic acid; 3-iodo-thyroacetic acid; 3,5-diiodo-L-thyronine; dextro-T4; thyronamine; 3-iodothyronamine; 3,3′,5-triiodothyronamine; 3,5-diiodothyronine; 3,5-dibromo-3′-isopropyl-L-thyronine; 3′-acetyl-3,5,diiodo-L-thyronine; a T3 conjugate, a T4 conjugate; 3,5-dimethyl-4-(4′-hydroxy-3′isopropylbenyl)-phenoxyacetic acid; a 5′-substituted analog of 3,5-dimethyl-4-(4′-hydroxy-3′isopropylbenyl)-phenoxyacetic acid; 3,5-dichloro-4-[(4-hydroxy-3-isopropylphenoxy)phenyl] acetic acid; 3,5-dimethyl-4-(4′-hydroxy-3′-benzyl) benzylphenoxyacetic acid; MGL-3196; a [1-(4-hyrodxy-benzyl)-1H-indol-5-yloxy]-acetic acid; a carboxylic acid analog; a 1-benzyl-4-aminoindole-based thyroid hormone analog; a T3-cholic acid conjugate; CGS 23425; 3,5-dibromo-3-pyridazinone-1-thyronine; (1R,4S)-4-(3-chlorophenyl)-2-((3,5-dimethyl-4-(4′-hydroxy-3′-isopropropylbenzyl)phenoxy)methyl)-2-oxido-(1,3,2)-disozaphophonane; MB07811; or a 1-benzylindole-based agonist.

5. The pharmaceutical composition of claim 1, further comprising an additional active agent.

6. The pharmaceutical composition of claim 5, wherein the additional active agent comprises iopanoic acid, iopanoate, ipodate, propylthiourea, propylthiouracil, 6-propylthiouracil, propranolol, D-propranolol, dexamethasone, cortisol, a glucocorticoid, amiodarone, desethlaminodarone, dronedarone, (3′),4′,4,6-(tetra)trihydroxyaurone, insulin, 3′,5′-cyclic adenosine monophosphate, butyrate, a phenolphthalein dye, or an environmental halogenated chemical.

7. The pharmaceutical composition of claim 5, wherein the additional active comprises a thyroid hormone derivative, a thyronine, a thyronamine, a thyroacetic acid, a chemically-modified thyroid hormone, a thyroid-hormone-linked macromolecule, or a thyroid hormone receptor agonist.

8. The pharmaceutical composition of claim 7, wherein the additional active agent comprises thyroxine; 3,3′,5-triiodothyronine; 3,5-dimethyl-3′-isoprophylthyronine; 3,5-dibromo-3′-pyridazinone-L-thyronine; 3,3′,5,5′ tetraiodoacetic acid; 3-iodo-thyroacetic acid; 3,5-diiodo-L-thyronine; dextro-T4; thyronamine; 3-iodothyronamine; 3,3′,5-triiodothyronamine; 3,5-diiodothyronine; 3,5-dibromo-3′-isopropyl-L-thyronine; 3′-acetyl-3,5,diiodo-L-thyronine; a T3 conjugate, a T4 conjugate; 3,5-dimethyl-4-(4′-hydroxy-3′isopropylbenyl)-phenoxyacetic acid; a 5′-substituted analog of 3,5-dimethyl-4-(4′-hydroxy-3′isopropylbenyl)-phenoxyacetic acid; 3,5-dichloro-4-[(4-hydroxy-3—isopropylphenoxy)phenyl] acetic acid; 3,5-dimethyl-4-(4′-hydroxy-3′-benzyl) benzylphenoxyacetic acid; MGL-3196; a [1-(4-hyrodxy-benzyl)-1H-indol-5-yloxy]-acetic acid; a carboxylic acid analog; a 1-benzyl-4-aminoindole-based thyroid hormone analog; a T3-cholic acid conjugate; CGS 23425; 3,5-dibromo-3-pyridazinone-1-thyronine; (1R,4S)-4-(3-chlorophenyl)-2-((3,5-dimethyl-4-(4′-hydroxy-3′-isopropropylbenzyl)phenoxy)methyl)-2-oxido-(1,3,2)-disozaphophonane; MB07811; or a 1-benzylindole-based agonist.

9. The pharmaceutical composition of claim 5, wherein the additional active agent comprises a glucocorticoid, a mineralocorticoid, a β-adrenergic agonist, a catecholamine, a growth factor, an inhibitor of a pro-inflammatory cytokine, an inhibitor of a pro-inflammatory chemokine, a compound that augments an anti-inflammatory cytokine, a compound that augments an anti-inflammatory chemokine, a compound that induces genetic expression of a β-adrenergic receptor, a surfactant lipid, or an apoprotein replacement therapy.

10. The pharmaceutical composition of claim 5, wherein the additional active agent comprises a salt of triiodothyronine (T3) or a salt of thyroxine (T4).

11. A pharmaceutical composition for administering directly to the lung of a subject, the composition comprising:
two or more active agents selected from:
a thyroid hormone, a deiodinase inhibitor, a thyroid hormone analog, a thyroid hormone mimetic, a glucocorticoid, a mineralocorticoid, a β-adrenergic agonist, a catecholamine, a growth factor, an inhibitor of a pro-inflammatory cytokine, an inhibitor of a pro-inflammatory chemokine, a compound that augments an anti-inflammatory cytokine, a compound that augments an anti-inflammatory chemokine, a compound that induces genetic expression of a β-adrenergic receptor, a surfactant lipid, or an apoprotein replacement therapy; and
a pharmaceutically acceptable buffer, adjusted to a pH of 5.5-8.5.

12. The pharmaceutical composition of claim 1, wherein the composition is aerosolized.

13. The pharmaceutical composition of claim 1, wherein the composition is nebulized.

14. A method for treating a subject having, or at risk of having inflammation of lung tissues, the method comprising:
administering to the subject an amount of the composition claim 1 effective to ameliorate lung inflammation, wherein the composition is administered directly to the pulmonary tract.

15. The method of claim 14, wherein the composition is administered by intratracheal instillation.

16. The method of claim 14, wherein the composition is administered by inhalation of an aerosolized formulation.

17. The method of claim 14, wherein the composition is administered by inhalation of a nebulized formulation.

18. The method of claim 14, wherein the total weight of the composition administered is a lung-delivered drug dose range of 10 ng to 5 mg.

19. The method of claim 14, wherein the composition is administered prior to the subject manifesting any symptom or clinical sign of lung inflammation.

20. The method of claim 14, wherein the composition is administered after the subject manifests a symptom or clinical sign of lung inflammation.

21. The method of claim 14, wherein the lung inflammation is a clinical sign of acute respiratory distress syndrome (ARDS).

22. The method of claim 14, wherein the lung inflammation is a symptom or clinical sign of premature birth, chest trauma, congestive heart failure, lung transplant, lung cancer radiotherapy, lung cancer chemotherapy, smoking, exposure to a pollutant, hypersensitivity pneumonitis, a reactive/obstructive lung disease, aspiration chemical pneumonitis/pneumonia, pneumonia, an infection of the nasosinus, intratracheal, intrabronchial or alveolar airspace, a connective tissue disease, Wegener's granulomatosis, Good pasture disease, acute eosinophilic pneumonia, chronic eosinophilic pneumonia, medication-related lung injury, cryptogenic organizing pneumonia, Churg-Strauss syndrome, congenital lung disease, COVID-19, or structural lung disease.

23. A method for treating a subject having, or at risk of pulmonary edema, the method comprising:
administering to the subject an amount of the composition of claim 1 effective to ameliorate pulmonary edema, wherein the composition is administered directly to the pulmonary tract.

24. The method of claim 23, wherein the composition is administered by intratracheal instillation.

25. The method of claim 23, wherein the composition is administered by inhalation of an aerosolized formulation.

26. The method of claim 23, wherein the composition is administered by inhalation of a nebulized formulation.

27. The method of claim 23, wherein the total weight of the composition administered is a lung-delivered drug dose range of 10 ng to 5 mg.

28. The method of claim 23, wherein the composition is administered prior to the subject manifesting any symptom or clinical sign of pulmonary edema.

29. The method of claim 23, wherein the composition is administered after the subject manifests a symptom or clinical sign of pulmonary edema.

30. The method of claim 23, wherein the pulmonary edema is a clinical sign of acute respiratory distress syndrome (ARDS).

31. The method of claim 23, wherein the pulmonary edema is a symptom or clinical sign of premature birth, chest trauma, congestive heart failure, lung transplant, lung cancer radiotherapy, lung cancer chemotherapy, smoking, exposure to a pollutant, hypersensitivity pneumonitis, a reactive/obstructive lung disease, aspiration chemical pneumonitis/pneumonia, pneumonia, an infection of the nasosinus, intratracheal, intrabronchial or alveolar airspace, a connective tissue disease, Wegener's granulomatosis, Good pasture disease, acute eosinophilic pneumonia, chronic eosinophilic pneumonia, medication-related lung injury, cryptogenic organizing pneumonia, Churg-Strauss syndrome, congenital lung disease, COVID-19, or structural lung disease.

32. The pharmaceutical composition of claim 11, wherein the composition is aerosolized.

33. The pharmaceutical composition of claim 11, wherein the composition is nebulized.
